SEQUENCE LISTING (51 sequences)

SEQ ID NO: 1 - DNA, 1836 bp, Rhizopus delemar
atgttatattcaaaagcatttcaatcttctttaaagaatgcaattaaacaaggtggtcgccgtcattatggcttgccatcctctgctattcatttaggcacagatccaagaaaagacaagacagttcaattgactcctctaagaggtgttaatattattcatgatcctcttctttcaaaaggtactgcatttagtctttcagaaagagaaagattgtctattcgtggtttagtaccacctcgttgtcaagaaatggacaaacaattacttcgtatcaaaagaaacttggatgcattagatacacccatagccaaatttgtctttttaactgcacttcaagatagaaatgagactttattctacaagttattaattacatatcttgaagaattagcaagtatcatttatacacctactgtgggccaagcttgtttattgtctcactctatctatcgtcgttctcgtggcatgtacttttcttcacaggacagaggtgccatgtcagctatggttcataactggcctcatgatgaagtagacgtcattgttgtcactgatggctctcgtgtcttgggcttgggtgatttaggtgccaatggtatgcaaattccgatcggtaaactcagcttgtacgtcgccgcgggtggtatccgaccacgttccgtcttgcctgtcgttttagacgttggcacaaacaatgaatctttgttacatgatcctctttatctcggtatgggtcatcctagattggatggtgaagaatattattcatttgtagatgagtgggtaactgctgttcaaagcagatggccaaatgcgcttattcaattcgaagatttcaaatatcctcacgcatataatttattaaacaaataccaaaacaaggtgacctgtttcaatgatgacattcagtctacttctgccatcactttagctggtcttcttgcatccttaaaagctcgtggtcgtcctcaagaatgtctttcagaggaacgcatcatctgtgtcggtgctggttcagccggtgtcggtgtctgtgaaggtatcattgatgccatggtctctcagggtaaggtagcctctcgtgaagaagcctattcccgtatttggatgttggatcagtgcggtctcctcggtaacccttccttaaccgatgatgctacagtcaatggcgaaagaacaactgaactagatgaacgtcaacgctgttacgtcaaatccgacttgccggatcgtctcaccttggaggccttgattgagcgtgtcaagccaaccgctatccttggcttgaccggtgttcctggtgtctttaccgaacaagccatccgtactatggccaaataccaagaaaaacccgtcgtattccctctttctaaccctgatacgcgtgctgaatgtaccgctgaagaagcattcaaatggacagaaggacgtgctatttttgcttctggcagtccctttgctgatgttcaacttcccaatggaaagatcggtagaaccaaccaatgtaataactcttacagtttccctggtcttggtcttggtatcactgtctctagagctacacgcgtgacacccaacatgtttttggaaaccgccaagactattgccaacatggcaagtccctctcaactcaaggagggcatcttattcccaggtgtcactcaccttcgtgatgttgccaaggccgttggtactcgtgtttgtgaagtagcttatgatgaaaatgtagctactgccgttttgaaagaaggtgaattgttgagtgaagtcgtacaaagctcgatgtttgaacctgaatatgttcctttggtccatgttcccaacatgcattag

SEQ ID NO: 2 - protein, 611 aa, Rhizopus delemar
MLYSKAFQSSLKNAIKQGGRRHYGLPSSAIHLGTDPRKDKTVQLTPLRGVNIIHDPLLSKGTAFSLSERERLSIRGLVPPRCQEMDKQLLRIKRNLDALDTPIAKFVFLTALQDRNETLFYKLLITYLEELASIIYTPTVGQACLLSHSIYRRSRGMYFSSQDRGAMSAMVHNWPHDEVDVIVVTDGSRVLGLGDLGANGMQIPIGKLSLYVAAGGIRPRSVLPVVLDVGTNNESLLHDPLYLGMGHPRLDGEEYYSFVDEWVTAVQSRWPNALIQFEDFKYPHAYNLLNKYQNKVTCFNDDIQSTSAITLAGLLASLKARGRPQECLSEERIICVGAGSAGVGVCEGIIDAMVSQGKVASREEAYSRIWMLDQCGLLGNPSLTDDATVNGERTTELDERQRCYVKSDLPDRLTLEALIERVKPTAILGLTGVPGVFTEQAIRTMAKYQEKPVVFPLSNPDTRAECTAEEAFKWTEGRAIFASGSPFADVQLPNGKIGRTNQCNNSYSFPGLGLGITVSRATRVTPNMFLETAKTIANMASPSQLKEGILFPGVTHLRDVAKAVGTRVCEVAYDENVATAVLKEGELLSEVVQSSMFEPEYVPLVHVPNMH

SEQ ID NO: 3 - DNA, 2298 bp, Rhizopus delemar
  trpC
atgaccactttacttattgacaactacgacagttttacttataatgtctatcaatacttgagctgccaaggcgccaatgtagttgtctacagaaacgacaaaatcaccatttccgaaattgagcaattggctcctcgcaatattgtcatctcacctggccctggccacccttccaccgatgccggtgtctctcgagaggccattcgagcttttgcaggaaagattcccatcttgggtatttgtatgggtcagcaatgtatgtatgaagtgtacggtggtaaagtgtcatatgcaggtgatattgtgcatggcaaggcatccagcatcaagcatgacagtcgaggtatcttcaagggcgttcctcaaaacaacatggtcactcgttaccattcccttgctggcatgccttctactttacctgaaacattagaagtcactgcgactaccgacgatggtatcatcatgggcattcgacacaaggaatacactgtcgaaggtgttcagttccatcctgaaagtatcctttgtgaacacggacatacgatgatcaacaacttcttaagcttgcgtggtggcacctgggaagagaatcctgcagccggtgttgtctttaagaaagctcgttccgaaacacccaaaatcagtgctagtgaatcccaactcgatctctctcagcaacaacctgccgcagcaccttccatcttgacccgcatttactctcaacgactcaaggatgttcaggcagccaaggagattcccggccagacatttgaagatttagaaaaacttttaaagttgcacgtcgccccacctcttcaagacgtcgtcgctcgcgtgcgtcaaagcaagcccgccttgatggccgaagtcaagcgtgcctctccctcgaaaggaaacattgatgtttcggccaacgcggctgagcaggcacttcaatatgctttagcaggtgcaagcgtcgtctctgttctgactgaacccaaatggttccgcggtacgattcatgatatgcatcaggtccgagaggccttgagccatctgcccaaccgtccttgtgtgttgagaaaggattttattgtcgatcgctatcaaatcttggaaggttgtctgtacggtgctgatactatcttgttgatcgtggccatgctgaatgatgaacaactgcacgaattgtatcactatgcgaaatcattaggtatggaacccttggtcgaagtcaataatacggaagagatggcccgtgccaatgctttgggcgcacgtctggtgggtgttaataatcgcaacttgcacagctttgatgttgatatggaaaccacgagtcgattggtagagatggtgcctgaaggaacgatcttgtgtgcactttctggtattactggacgagctgatgttgaaatgtacgtcaaacagggtgtgcacgctgtcttggtgggtgaagccctgatgcgtgcttggaatttgaaggagtttgtgtctgatttgttgggtcatgaaaagaaggatcctgtgcctgtgtccaaggaatcaaaatcttcactagtcaaggtatgtggtatctctagtgtggatgcagcagttgaagcagccaagtcaggggctgacttgattggtcttatctttgctgaaaagtccaaacgaaaagtgtctttggaaagagctcaagaaatcgtgtcctcagtgcgtgcgttggatattcaagtcaaacgaacgttatcaaatgatgattctcaactggattggttccagatgcacaagcgtctcttggaaaagcgagcaagaaaacctttggtagttggcgtgtttgtgaatcaatcgattgaatacatgactgaggtggcaacgacagtcggactggaccttattcagctgcatggaaccgaatcaacggagcttgcacgctatttacccgtgcctgtcatcaaagctttccatatcgacagtggtgagttcaatgaagctcagataccaaacctaaatcaaccaggctcttatcattatgtcttactggacgctaaagtgcccagcttaccatcggatcaacaaggtggacgtggtgtcaagtttgattggtcaattgctaccaaaatcgtgaaacataggcactttgagtttttgggtaatcaagatttccctgtcatcttggctggtgggttggatcctaccaatgtggcatctgccattcaacaggtgaaaccctggattgtggatgtgtcgagtggtgttgaaacagatggagtgaaggatttagaaaagattcgtgcctttgttaaaactgtccagtcaacacaattttaa

SEQ ID NO: 4 - DNA, 1000 bp, Rhizopus delemar
  ADH promoter
tagagggaaaaagagagaattgaaataggagaggatgagtcaaaatatagtttacataaaatttctcttttttgtgttaaatataatctaatagcaggggttttcttagtttacgtttatatcaaagttatcaagcatacacttttttatgatttttcatactttaatcccttttagtattctattgtttgaaaggagagaaaaaacagctgagggtacggtgcacacgagatcttacgataattttcctgcccaacaggaaagaagtaattgatcttgattgacgctcggagtttgcacgttcggagtttgcacttcacattgagttatactcttacttattttgaaggaagggacgagaaaagatgtaaatataataataacagtagtaaatagtatgcgcatcaagaacagctaccaacaaaagagagaaatatgagcttaataatgaacaatgtaaatggcagaatgaaatttaattatcaaagcggcatctttcagaccttccgttacttccgatagagttttttatgcaaagtaataacaactgtatatataaaaaaaagaaggttatcaagcaaaagccacaatgtcatatctggaataatcaagagtaactattgaatgttggtagccaaaagaggcacgtaattttatgacgaaatatcacacaaaaagattattttgacaattcatgaataggacagagatacaccctaaacatgaaatgtaagctatatttaaacacctcaagttaattttgaagcttcatttgtattattgtaaccatttagacaagctaaatcctttttattattgtccttattgattttatccagattaccgtatctaaagagcgatcaacagaaaaacggctgattttagaccaaagtttcacaaactacatttgcatgaacgtcatatatatataaaccttgacttttcttttttttttttttttttttttttcattatcaattaatacaattaaataacaaaa

SEQ ID NO: 5 - DNA, 634 bp, Rhizopus delemar
  ADH terminator
tcattttaattacgcattttcatttactaatttgttacattttgataacgtcaataaatcttctaatttcttttgttctcaaacagtttacagttctatctttttttttaccacaatcaattctcaatatacaacatagcaaatgtgcttcagtaaattcattaaattcttttaaaaaaaggtaatttgtagcataaaattcgactttattgacgttttttttatgatcatatacaaataaaatagttgcgaatgagaactaaatttttcattgtttttagtcatatcatctggctgttgcacgatgatcgcagcatatttttcttcacaacactcatcctataagcacctttcaggactttcgtctgcactttccatatttgatttcatcaattgatttgaatttttatccagtacaatggtttgaatctatacaataaattagtcacagtataaaattatgtctcatcttgaacacacacctgcttaacaaagaaatgaagcactctatcaatagtaaatacaatatatgcatcgatgccaaatatatatcgtacattctcttcaaacgtagcttgatctaaatcgccatcaataaacctttcaatcatcctcactagtgcattataatggc

SEQ ID NO: 6 - DNA, 1735 bp, Rhizopus delemar
  pdc
atgcctgctattaaaatcggtcaacatctccttaaccgtcttaaggaaatcaacattgatgttgtctttggtgttcctggtgatttcaacatggtaagcagacaattgaattgaacgagagcctataaacttattatttctatagcccttgttggatatcattgaagatgacccagaacttacctggggtaacaatgccaacgaattgaatgcatcttatgcagctgatggttatgctcgtattcgtggtgcaggtgctgttgtcactacctttggtgtaggtgagctgtctgctgtcaacggtattgctggttcatactctgagatgcttcccgtgattcacatcgtcggtactccttctactaaatcccaagctgccggtgccatgcttcaccactctttgggtgacggtaactttgatgtgttcttcaacatgtcctccatgattgcctgtgcctctgctcacctcaagaaacaaacggccattgcagaaattgaccgtgtgatctcccaagctgttctctccaagcgtacaggttacattggtatccctatcgatctgatcaagactgaggttgaagtacctgagcccattcctgccctcaagaccgaattacccaaaaacccagctgatgtccaagcgattgccttgagagtggtcacggatgcgatcgccaaagcccaattccctgtgattgttgtcgatggctgtgtgcttcgccagagatgccaaaaggcagtacaagcctttatcgaacgtactggtttccctacttatgttgctcctatgggtaagggtgccgttgacgaatcctctgtgagttaccgtggctgctactcgggtaatgtcacattggaagcagtgaatgaagagatcaagcaagccgatttgatcatcgaagtgggctccatcaagtctgatttcaacacgggtaacttttcatactctctcgaccgttccaagacgatcaccttgcactcctttgccaccatcgtgttttgtgctgaataccaaaaggtctccatgctcgaattcattcctctcttgacccaagcccttcccgaacaaccccgtcaattcaacctgggtccccgcccaagacccgtacctatccaacccggtaccgaaatcacccacaactacttttggcacaaggtacccgaattcatggatgagaacgccattgtctgtgccgagaccggtacagctgaatttgcttcactcaacatggacggacccaagggaacgacttatatcacccaattcctctggggctctatcggtttctcagtaggtgccgctgtgggtgctgcgatcgccgctcgtgatcgtcgtgtgtatctctttgtcggtgatggttccttccaattgacctgtcaagaaatctctggcttccttcgccatggtttgacacctgtgatcttcttgctgaacaatgacggttacttgatcgaaaaactcattcacggtcccgaacgtgcctataataactttcaaatgtgggaatacagcaagacgcttgattatttcggtgctcatcttgaacacaacaagtccatgggtgttcctcccgttggcttcgaaggcaaggtagccacacgcgatgaatttgaatccgccatgagacaggttcaagccaatcctgacaagattcatttccttgaagtcattatgcctcaatttgactctcctcgtgaacttgaactcttggttgccaactctgaaaaccgttaa

SEQ ID NO: 7 - DNA, 3006 bp, Artificial
  LeftTALEN-pdc
atggactacaaagaccatgacggtgattataaagatcatgacatcgattacaaggatgacgatgacaagatggcccccaagaagaagaggaaggtgggcattcaccgcggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacttgaccccagaccaggtagtcgcaatcgcgaacaataatgggggaaagcaagccctggaaaccgtgcaaaggttgttgccggtcctttgtcaagaccacggcctgactcccgatcaagttgtagcgattgcgtcgcatgacggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgaccccagaccaggtagtcgcaatcgcgtcaaacggagggggaaagcaagccctggaaaccgtgcaaaggttgttgccggtcctttgtcaagaccacggcctgaccccagaccaggtagtcgcaatcgcgaacaataatgggggaaagcaagccctggaaaccgtgcaaaggttgttgccggtcctttgtcaagaccatggcctgactcccgatcaagttgtagcgattgcgtcgcatgacggagggaaacaagcattggagactgtccaacggctccttcccgtgttgtgtcaagcccacggtttgacgcctgcacaagtggtcgccatcgccagcaatggcggcggtaagcaggcgctggaaacagtacagcgcctgctgcctgtactgtgccaggatcatggactgaccccagaccaggtagtcgcaatcgcgtcgaacattgggggaaagcaagccctggaaaccgtgcaaaggttgttgccggtcctttgtcaagaccacggcctgaccccagaccaggtagtcgcaatcgcgtcaaacggagggggaaagcaagccctggaaaccgtgcaaaggttgttgccggtcctttgtcaagaccacggcctgactcccgatcaagttgtagcgattgcgtccaacggtggagggaaacaagcattggagactgtccaacggctccttcccgtgttgtgtcaagcccatggattgaccccagaccaggtagtcgcaatcgcgtcaaacattgggggaaagcaagccctggaaaccgtgcaaaggttgttgccggtcctttgtcaagaccacggcctgactcccgatcaagttgtagcgattgcgtcgaacattggagggaaacaagcattggagactgtccaacggctccttcccgtgttgtgtcaagcccacggtttgacgcctgcacaagtggtcgccatcgccagcaatattggcggtaagcaggcgctggaaacagtacagcgcctgctgcctgtactgtgccaggatcatggactgaccccagaccaggtagtcgcaatcgcgtcaaacattgggggaaagcaagccctggaaaccgtgcaaaggttgttgccggtcctttgtcaagaccacggcctgaccccagaccaggtagtcgcaatcgcgtcaaacggagggggaaagcaagccctggaaaccgtgcaaaggttgttgccggtcctttgtcaagaccacggcctgactcccgatcaagttgtagcgattgcgtcgcatgacggagggaaacaagcattggagactgtccaacggctccttcccgtgttgtgtcaagcccacggtctgacacccgaacaggtggtcgccattgctaataataacggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaacttttaa

SEQ ID NO: 8 - DNA, 3006 bp, Artificial
  RightTALEN-pdc
atggactacaaagaccatgacggtgattataaagatcatgacatcgattacaaggatgacgatgacaagatggcccccaagaagaagaggaaggtgggcattcaccgcggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggcgctgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccttgaacttgaccccagaccaggtagtcgcaatcgcgtcaaacggagggggaaagcaagccctggaaaccgtgcaaaggttgttgccggtcctttgtcaagaccacggcctgactcccgatcaagttgtagcgattgcgaataacaatggagggaaacaagcattggagactgtccaacggctccttcccgtgttgtgtcaagcccacggtttgacgcctgcacaagtggtcgccatcgccagcaatattggcggtaagcaggcgctggaaacagtacagcgcctgctgcctgtactgtgccaggatcatggactgaccccagaccaggtagtcgcaatcgcgtcaaacggagggggaaagcaagccctggaaaccgtgcaaaggttgttgccggtcctttgtcaagaccacggcctgaccccagaccaggtagtcgcaatcgcgtcaaacggagggggaaagcaagccctggaaaccgtgcaaaggttgttgccggtcctttgtcaagaccacggcctgactcccgatcaagttgtagcgattgcgtcgaacggtggagggaaacaagcattggagactgtccaacggctccttcccgtgttgtgtcaagcccacggtttgacgcctgcacaagtggtcgccatcgccagccatgatggcggtaagcaggcgctggaaacagtacagcgcctgctgcctgtactgtgccaggatcatggactgaccccagaccaggtagtcgcaatcgcgtcacatgacgggggaaagcaagccctggaaaccgtgcaaaggttgttgccggtcctttgtcaagaccacggcctgaccccagaccaggtagtcgcaatcgcgtcaaacggagggggaaagcaagccctggaaaccgtgcaaaggttgttgccggtcctttgtcaagaccacggcctgactcccgatcaagttgtagcgattgcgtccaacggtggagggaaacaagcattggagactgtccaacggctccttcccgtgttgtgtcaagcccatggattgaccccagaccaggtagtcgcaatcgcgtcaaacattgggggaaagcaagccctggaaaccgtgcaaaggttgttgccggtcctttgtcaagaccacggcctgactcccgatcaagttgtagcgattgcgtcgaacattggagggaaacaagcattggagactgtccaacggctccttcccgtgttgtgtcaagcccacggtttgacgcctgcacaagtggtcgccatcgccaacaacaacggcggtaagcaggcgctggaaacagtacagcgcctgctgcctgtactgtgccaggatcatggactgaccccagaccaggtagtcgcaatcgcgtcaaacattgggggaaagcaagccctggaaaccgtgcaaaggttgttgccggtcctttgtcaagaccacggcctgaccccagaccaggtagtcgcaatcgcgtcacatgacgggggaaagcaagccctggaaaccgtgcaaaggttgttgccggtcctttgtcaagaccatggcctgactcccgatcaagttgtagcgattgcgaataacaatggagggaaacaagcattggagactgtccaacggctccttcccgtgttgtgtcaagcccacggtctgacacccgaacaggtggtcgccattgctaataataacggaggacggccagccttggagtccatcgtagcccaattgtccaggcccgatcccgcgttggctgcgttaacgaatgaccatctggtggcgttggcatgtcttggtggacgacccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaacttttaa

SEQ ID NO: 9 - DNA, 3735 bp, Rhizopus delemar
  TALEN binding region
gcagacttcaacagttggcttttttaagtaaaaaagatagtgtttctcgataaattaattttgataagataaagagagagagagaatgaaacgatactttttgtttattttttcgtgaataaaaaaaagtcattccacttttagaaaccaagagtagtgttcttgacaacaacgccatttttttgcgtagtcttaaaaaaaaagtcattcgtttttgaaggaatacaacggatctgattaaggaaggcagtagattcaatttgattggataaagttcatcttccgaaatcgacttttatgtgtcattgtatttgggcgcataaaagtaataacgggatgttccactgcgttcggttatcagttttaatatcagaaatcatacatgtttttttttatattacttatcccgaaatatctattgtctcctccgtaggctgacttgctgtcaatgtcaaataaggtatggctgtagtacattctatgcacgttctataaagtaatttactagtttaaagcaaaaaacatgagcaaaagaagcctgatatgctttggttttcatttcatttgaacgtattctttctttttttttttgcagagtattttttatgcgtttttttttttttttttttttttttactacgatcaaagatatcttcgattacagaaacagaaagggatttgccggagtatgaatacttgtcttttaattttattctttgaaagaagcataaagaataaactgatggtacggtgtttatctgttcacaagtactggtaagcaacatacttgttttaaggaaacattaaactctatttgccagctattgaaagaaagaaagaaaaaatatgcattttaattctccgatgctttcgggcacagtcgttattaggtatcgatttgctgatatgacattgcgacgaaaatagtataaaagaaacccactcttttttcgttcaaaaaaaatcattattcaatactctacaaaacaaatttaaaaacatgcctgctattaaaatcggtcaacatctccttaaccgtcttaaggaaatcaacattgatgttgtctttggtgttcctggtgatttcaacatggtaagcagacaattgaattgaacgagagcctataaacttattatttctatagcccttgttggatatcattgaagatgacccagaacttacctggggtaacaatgccaacgaattgaatgcatcttatgcagctgatggttatgctcgtattcgtggtgcaggtgctgttgtcactacctttggtgtaggtgagctgtctgctgtcaacggtattgctggttcatactctgagatgcttcccgtgattcacatcgtcggtactccttctactaaatcccaagctgccggtgccatgcttcaccactctttgggtgacggtaactttgatgtgttcttcaacatgtcctccatgattgcctgtgcctctgctcacctcaagaaacaaacggccattgcagaaattgaccgtgtgatctcccaagctgttctctccaagcgtacaggttacattggtatccctatcgatctgatcaagactgaggttgaagtacctgagcccattcctgccctcaagaccgaattacccaaaaacccagctgatgtccaagcgattgccttgagagtggtcacggatgcgatcgccaaagcccaattccctgtgattgttgtcgatggctgtgtgcttcgccagagatgccaaaaggcagtacaagcctttatcgaacgtactggtttccctacttatgttgctcctatgggtaagggtgccgttgacgaatcctctgtgagttaccgtggctgctactcgggtaatgtcacattggaagcagtgaatgaagagatcaagcaagccgatttgatcatcgaagtgggctccatcaagtctgatttcaacacgggtaacttttcatactctctcgaccgttccaagacgatcaccttgcactcctttgccaccatcgtgttttgtgctgaataccaaaaggtctccatgctcgaattcattcctctcttgacccaagcccttcccgaacaaccccgtcaattcaacctgggtccccgcccaagacccgtacctatccaacccggtaccgaaatcacccacaactacttttggcacaaggtacccgaattcatggatgagaacgccattgtctgtgccgagaccggtacagctgaatttgcttcactcaacatggacggacccaagggaacgacttatatcacccaattcctctggggctctatcggtttctcagtaggtgccgctgtgggtgctgcgatcgccgctcgtgatcgtcgtgtgtatctctttgtcggtgatggttccttccaattgacctgtcaagaaatctctggcttccttcgccatggtttgacacctgtgatcttcttgctgaacaatgacggttacttgatcgaaaaactcattcacggtcccgaacgtgcctataataactttcaaatgtgggaatacagcaagacgcttgattatttcggtgctcatcttgaacacaacaagtccatgggtgttcctcccgttggcttcgaaggcaaggtagccacacgcgatgaatttgaatccgccatgagacaggttcaagccaatcctgacaagattcatttccttgaagtcattatgcctcaatttgactctcctcgtgaacttgaactcttggttgccaactctgaaaaccgttaaaatcttagaattcattttttttttgtatcattcgttctatacttcattactacagtaaattcataataaaatcatatttttgaaaataagctttttttttcttttacctgtgggccccatgatgattggtgtaacttttacaagctttggattgccaagttttatcagcatgtgacaagtcagcacacgctcttgaatatttctaggcaacaaggctttgatttcttttagggacttggtttatattgaatccttttaaggattgtatttaaaaatagttttattgcaagaaatgatgtatgtatgggtgtttgttacaatgtaaataggcatgaaaatatatacaacaagaaaaaacggtctataaagtctataagcattcaataaaatgaattattattcgtcatggccacaaggacaagattcatcgtgagtgaactgagagacatcgattccacaaccaggacagaaggcatattcataaagaaatgtaaattgaaccaagtttagtgagtccatgtttttgattgtaaaaggataaggaaaagaaagagacagtttgagaagcaacctttttataaacatattccttcaaccaaggtcattcattccctaaactattttccttgtgtggcaaaagtacgccggcagagacggataaatatgcttgcgctctcttttgacttgtacaactctaatttgtttgtgctaagcatcgatgggggtccacttttagccattgtttgttataggccttggcattgtctattaataaattagaaaaagagcagacgagagagagagagagagagtgaagggggggggaccatctgatctgtctttacaagtcacatctatgacaatttttatgccttttttgagaaaaaaaaatctattttttttatccttccttcttactctttcacagcatgtcccatcttccgaccatcgtcgtcctcatttctggatccggcaccaaccttcaagctcttattgacgc

SEQ ID NO: 10 - DNA, 18 bp, Rhizopus delemar
  Target of LeftTALEN-pdc
tgcctgctattaaaatcg

SEQ ID NO: 11 - DNA, 18 bp, Rhizopus delemar
  Target of RightTALEN-pdc
ttgatttccttaagacgg

SEQ ID NO: 12 - DNA, 46 bp, Artificial
  primer oJK162
cgagctcgaattatttaaatgaacagcaagttaataatctagaggg

SEQ ID NO: 13 - DNA, 39 bp, Artificial
  primer oJK163
tatgaccatgattacgatgagaggcaaaatgaagcgtac

SEQ ID NO: 14 - DNA, 30 bp, Artificial
  primer oJK164
atttaaataattcgagctcggtacccgggg

SEQ ID NO: 15 - DNA, 20 bp, Artificial
  primer oJK165
cgtaatcatggtcatagctg

SEQ ID NO: 16 - DNA, 29 bp, Artificial
  primer oJK202
tagagggaaaaagagagaattgaaatagg

SEQ ID NO: 17 - DNA, 31 bp, Artificial
  primer oJK204
ttttgttatttaattgtattaattgataatg

SEQ ID NO: 18 - DNA, 36 bp, Artificial
  primer oJK205
aattaaataacaaaatcattttaattacgcattttc

SEQ ID NO: 19 - DNA, 37 bp, Artificial
  primer oJK216
catgattacgcggccgcgccattataatgcactagtg

SEQ ID NO: 20 - DNA, 38 bp, Artificial
  primer oJK210
ctctttttccctctaatgagaggcaaaatgaagcgtac

SEQ ID NO: 21 - DNA, 44 bp, Artificial
  primer oJK211
aattaaataacaaaaatgtcttctatcgaaacctccaaaatctc

SEQ ID NO: 22 - DNA, 35 bp, Artificial
  primer trpC-lost-F
tttaaattagagggaaaaagagagaattgaaatag

SEQ ID NO: 23 - DNA, 35 bp, Artificial
  primer trpC-lost-R
tccctctaatttaaatgaattcgagctcggtaccc

SEQ ID NO: 24 - DNA, 37 bp, Artificial
  primer NK-141
aattaaataacaaaaatgttatattcaaaagcatttc

SEQ ID NO: 25 - DNA, 33 bp, Artificial
  primer NK-163
gcgtaattaaaatgactaatgcatgttgggaac

SEQ ID NO: 26 - DNA, 25 bp, Artificial
  primer NK-011
ttttgttatttaattgtattaattg

SEQ ID NO: 27 - DNA, 21 bp, Artificial
  primer NK-012
tcattttaattacgcattttc

SEQ ID NO: 28 - DNA, 34 bp, Artificial
  primer pUC18-Pae1-F3
ctgcaggtcgactctagaggatccccgggtaccg

SEQ ID NO: 29 - DNA, 35 bp, Artificial
  primer pUC18-Hind3-R3
gcttggcactggccgtcgttttacaacgtcgtgac

SEQ ID NO: 30 - DNA, 45 bp, Artificial
  primer PDC1-upstr-F
cggccagtgccaagcgcagacttcaacagttggcttttttaagta

SEQ ID NO: 31 - DNA, 47 bp, Artificial
  primer PDC1-upstr-R
cattttgcctctcatgtttttaaatttgttttgtagagtattgaata

SEQ ID NO: 32 - DNA, 29 bp, Artificial
  primer trpCpro-R
gaacagcaagttaataatctagagggcgc

SEQ ID NO: 33 - DNA, 29 bp, Artificial
  primer trpCter-F
atgagaggcaaaatgaagcgtacaaagag

SEQ ID NO: 34 - DNA, 49 bp, Artificial
  primer PDC1-downstr-F
attaacttgctgttcaatcttagaattcattttttttttgtatcattcg

SEQ ID NO: 35 - DNA, 49 bp, Artificial
  primer PDC1-downstr-R
agagtcgacctgcaggcgtcaataagagcttgaaggttggtgccggatc

SEQ ID NO: 36 - DNA, 29 bp, Artificial
  primer oJK899
gaacagcaagttaataatctagagggcgc

SEQ ID NO: 37 - DNA, 29 bp, Artificial
  primer oJK900
aatcttagaattcattttttttttgtatc

SEQ ID NO: 38 - DNA, 44 bp, Artificial
  primer oJK901
attaacttgctgttctagagggaaaaagagagaattgaaatagg

SEQ ID NO: 39 - DNA, 33 bp, Artificial
  primer NK-195
atgaattctaagattctaatgcatgttgggaac

SEQ ID NO: 40 - DNA, 30 bp, Artificial
  primer oJK902
gcagacttcaacagttggcttttttaagta

SEQ ID NO: 41 - DNA, 34 bp, Artificial
  primer oJK903
gcgtcaataagagcttgaaggttggtgccggatc

SEQ ID NO: 42 - DNA, 30 bp, Artificial; identical to SEQ ID NO: 40
  primer PDC1-upstr-F2

SEQ ID NO: 43 - DNA, 34 bp, Artificial; identical to SEQ ID NO: 41
  primer PDC1-downstr-R-P

SEQ ID NO: 44 - DNA, 31 bp, Artificial; identical to SEQ ID NO: 17
  primer adhpro-R

SEQ ID NO: 45 - DNA, 27 bp, Artificial
  primer adhter-F
tcattttaattacgcattttcatttac

SEQ ID NO: 46 - DNA, 40 bp, Artificial
  primer adhpro-TALEN-F
aattaaataacaaaaatggactacaaagaccatgacggtg

SEQ ID NO: 47 - DNA, 40 bp, Artificial
  primer TAELN-adhter-R
gcgtaattaaaatgattaaaagtttatctcgccgttatta

SEQ ID NO: 48 - DNA, 45 bp, Artificial
  primer adhpro-exo1-F
aattaaataacaaaaatgaaaatccaagttgcttctcctattgac

SEQ ID NO: 49 - DNA, 46 bp, Artificial
  primer exo1-adhter-R
gcgtaattaaaatgattatcttctttcatgagaaacactaaacttg

SEQ ID NO: 50 - DNA, 24 bp, Artificial
  primer NK-069
cagaaaaacggctgattttagacc

SEQ ID NO: 51 - DNA, 27 bp, Artificial
  primer NK-118
ctgtagtaatgaagtatagaacgaatg

(19) United States
(12) Patent Application Publication
(10) Pub. No.: US 2019/0194700 A1
(43) Pub. Date: Jun. 27, 2019

(54) Mutant Filamentous Fungus and Method for Producing C4 Dicarboxylic Acid Using Same

(71) Applicant: Kao Corporation, Chuo-ku, Tokyo (JP)

(72) Inventor: Kyoshiro NONAKA, Wakayama-shi, Wakayama (JP)

(73) Assignee: Kao Corporation, Chuo-ku, Tokyo (JP)

(21) Appl. No.: 16/330,173

(22) PCT Filed: Sep. 5, 2017
(86) PCT No.: PCT/JP2017/031857

§ 371 Date: Mar. 4, 2019

(30) Foreign Application Priority Data
Sep. 15, 2016 (JP) 2016-180627

(51) Int. Cl. C12P 7/46; C12N 1/14; C12R 1/845

(52) U.S. Cl. CPC C12P 7/46; C12R 1/845; C12N 1/14

(57) ABSTRACT

Provided is a mutant filamentous fungus having improved C4 dicarboxylic acid productivity and a method for producing a C4 dicarboxylic acid using the mutant filamentous fungus. A mutant filamentous fungus having enhanced expression of at least one polypeptide selected from the group consisting of a polypeptide consisting of the amino acid sequence represented by SEQ ID No: 2; a polypeptide consisting of an amino acid sequence having an identity of at least 90% with the amino acid sequence represented by SEQ ID No: 2 and having malic enzyme activity; and a polypeptide consisting of an amino acid sequence having deletion, substitution, addition or insertion of one or more amino acids with respect to the amino acid sequence represented by SEQ ID No: 2 and having malic enzyme activity.

### FIELD OF THE INVENTION

The present invention relates to a mutant filamentous fungus and a method for producing a C4 dicarboxylic acid using the same.

### BACKGROUND OF THE INVENTION

C4 dicarboxylic acids are utilized not only in various applications in the food industry as an acidulant, an antimicrobial agent and a pH adjusting agent, but also used as a raw material for synthetic resins and biodegradable polymers. Thus, C4 dicarboxylic acids are industrially valuable substances. C4 dicarboxylic acids are industrially produced by either chemical synthesis from petrochemical raw materials or microbial fermentation. Previously, C4 dicarboxylic acids have been mainly produced by chemical synthesis due to a lower cost. However, from the viewpoint of rising costs of the raw materials, the burden on the environment, and the like, production methods by microbial fermentation using a recyclable resource as a raw material have recently been attracting attention.

It is known that fumaric acid, which is one of C4 dicarboxylic acids, can be produced by using a fermentative fungus, such as Rhizopus. Rhizopus produces fumaric acid using glucose as a carbon source, and excretes the produced fumaric acid to the outside of the cell. To date, as techniques for producing fumaric acid with high productivity by using Rhizopus, improvements of culturing methods, and preparations of strains having high productivity by mutation breeding are known. However, since the genetic background of Rhizopus has not yet been well studied, the development of the techniques for producing fumaric acid with high productivity by Rhizopus through gene recombination is not easy and has little information. There are only a few reports for improving fumaric acid productivity by introducing a gene encoding pyruvate carboxylase from Saccharomyces cerevisiae into Rhizopus delemar (Patent Literature 1), or by introducing a gene encoding phosphoenolpyruvate carboxylase from B. coli into Rhizopus oryzae (Non Patent Literature 1).

Malic enzyme (ME) is an enzyme catalyzing oxidative decarboxylation of malic acid coupled with reduction of NAD⁺ or NADP⁺ to produce pyruvic acid and CO₂. ME is found in the cytoplasm of B. coli, and also in the genus Streptococcus, Candida, Bradyrhizobium, Corynebacterium and lipid-producing fungi such as oleaginous yeast, Mucor circinelloides and Mortierella alpine. ME is reported to be involved in various metabolic pathways such as photosynthesis, lipid synthesis and energy metabolism pathway. Non Patent Literature 2 reports that succinic acid production was increased in B. coli to which a malic enzyme gene sfcA was introduced. Patent Literature 2 reports that malic acid production was increased in E. coli in which a fum gene was knocked out and a malic enzyme gene was allowed to overexpress. However, the role of ME in the metabolic pathway of filamentous fungi such as Rhizopus is still unknown.
(Patent Literature 1) Chinese Patent Publication No. CN103013843
(Patent Literature 2) Chinese Patent No. 101255405
(Non Patent Literature 1) Metabolic Engineering, 2012, 14: 512-520
(Non Patent Literature 2) Biotech. Bioeng., 2001, 74: 89-95

### SUMMARY OF THE INVENTION

In one aspect, the present invention provides a mutant filamentous fungus having enhanced expression of at least one polypeptide selected from the group consisting of the followings:
a polypeptide consisting of the amino acid sequence represented by SEQ ID No: 2,
a polypeptide consisting of an amino acid sequence having an identity of at least 90% with the amino acid sequence represented by SEQ ID No: 2 and having malic enzyme activity, and
a polypeptide consisting of an amino acid sequence having deletion, substitution, addition or insertion of one or more amino acids with respect to the amino acid sequence represented by SEQ ID No: 2 and having malic enzyme activity.

In another aspect, the present invention provides a method for producing a C4 dicarboxylic acid, comprising culturing the mutant filamentous fungus.

In another aspect, the present invention provides a method for producing a mutant filamentous fungus, comprising, in a host filamentous fungus, enhancing expression of at least one polypeptide selected from the group consisting of the following:
a polypeptide consisting of the amino acid sequence represented by SEQ ID No: 2,
a polypeptide consisting of an amino acid sequence having an identity of at least 90% with the amino acid sequence represented by SEQ ID No: 2 and having malic enzyme activity, and
a polypeptide consisting of an amino acid sequence having deletion, substitution, addition or insertion of one or more amino acids with respect to the amino acid sequence represented by SEQ ID No: 2 and having malic enzyme activity.

In another aspect, the present invention provides a method for improving C4 dicarboxylic acid productivity, in a filamentous fungus, comprising enhancing expression of at least one polypeptide selected from the group consisting of the followings:
a polypeptide consisting of the amino acid sequence represented by SEQ ID No: 2,
a polypeptide consisting of an amino acid sequence having an identity of at least 90% with the amino acid sequence represented by SEQ ID No: 2 and having malic enzyme activity, and
a polypeptide consisting of an amino acid sequence having deletion, substitution, addition or insertion of one or more amino acids with respect to the amino acid sequence represented by SEQ ID No: 2 and having malic enzyme activity in the filamentous fungus.

### DETAILED DESCRIPTION OF THE INVENTION

The present invention relates to a mutant filamentous fungus having improved C4 dicarboxylic acid productivity and a method for producing a C4 dicarboxylic acid using the mutant filamentous fungus.

The present inventor has conducted intensive studies, and found that a filamentous fungus in which expression of a polypeptide consisting of a predetermined amino acid sequence and having malic enzyme activity has been enhanced has improved C4 dicarboxylic acid productivity.

According to the present invention, a mutant filamentous fungus having improved C4 dicarboxylic acid productivity and a production method thereof are provided. The mutant filamentous fungus of the present invention is useful for biological production of a C4 dicarboxylic acid. According to the method for producing a C4 dicarboxylic acid using the mutant filamentous fungus of the present invention, it is possible to highly efficiently produce a C4 dicarboxylic acid. The features and advantages of the present invention mentioned above (including those not mentioned above) will be more clearly understood based on the following description of the specification.

### 1. Definition

In the specification, the identity of amino acid sequences or nucleotide sequences is calculated in accordance with the Lipman-Pearson method (Science, 1985, 227: 1435-1441). Specifically, the identity is calculated by analysis using the homology analysis program of genetic information processing software, GENETY Ver. 12, and assigning 2 to the Unit size to compare (ktup).

In the specification, “an identity of at least 90%” regarding an amino acid sequence or a nucleotide sequence refers to an identity of 90% or more, preferably 95% or more, more preferably 96% or more, further preferably 97% or more, further preferably 98% or more, further preferably 99% or more.

In the specification, the “amino acid sequence having deletion, substitution, addition or insertion of one or more amino acids” refers to an amino acid sequence in which 1 or more and 30 or less, preferably 1 or more and 10 or less, more preferably 1 or more and 5 or less, further preferably 1 or more and 3 or less amino acid(s) is(are) deleted, substituted, added or inserted. In the specification, the “nucleotide sequence in which one or more nucleotides are deleted, substituted, added or inserted” refers to a nucleotide sequence in which 1 or more and 90 or less, preferably 1 or more and 30 or less, more preferably 1 or more and 15 or less, further preferably 1 or more and 9 or less nucleotide(s) is(are) deleted, substituted, added or inserted. In the specification, “addition” of an amino acid or a nucleotide includes addition of the amino acid or the nucleotide to one and both ends of a sequence.

In the specification, “upstream” and “downstream” regarding a gene refer to upstream and downstream of the gene in the transcriptional orientation. For example, “a gene arranged downstream of a promoter” means that the gene is present on the 3′ side of the promoter in a DNA sense strand and the upstream of a gene means the region on the 5′ side of the gene in the DNA sense strand.

In the specification, the “operable linking” between a regulatory region and a gene refers to the linking of the gene to the regulatory region such that the gene can be expressed under control of the regulatory region. A procedure for “operable linking” between a gene and a regulatory region is well known to those skilled in the art.

In the specification, the term “originally” used for function, property and trait of a microorganism means that the function, property and trait are present in the microorganism of a wild type. In contrast, the term “exogenous” is used for representing that the function, property and trait are not originally present in the microorganism but externally introduced in the microorganism. For example, an “exogenous” gene or polynucleotide is a gene or polynucleotide introduced in a microorganism from the outside. The exogenous gene or polynucleotide may be derived from a homogeneous biological species of the microorganism in which the gene or polynucleotide is introduced or a different biological species (more specifically, heterologous gene or polynucleotide).

In the specification, the “C4 dicarboxylic acid productivity” of a microorganism is represented as a production speed of a C4 dicarboxylic acid in a culture medium of the microorganism; more specifically, a value (g/L/h) obtained by dividing the mass of the C4 dicarboxylic acid per medium volume produced by the microorganism during a certain culture time elapsed after the start of culture, by culture time. The amount of a C4 dicarboxylic acid produced by a microorganism can be calculated as the amount of the C4 dicarboxylic acid in the culture supernatant, which is obtained by removing cells from a cultured broth of the microorganism. The amount of a C4 dicarboxylic acid in the culture supernatant can be measured by high performance liquid chromatography (HPLC). The measurement procedure will be more specifically described later in Reference Example 1.

In the specification, “improvement of C4 dicarboxylic acid productivity” in a mutant means that C4 dicarboxylic acid productivity of the mutant has been improved compared to that of a host or a control. The improvement rate of C4 dicarboxylic acid productivity in a mutant is calculated in accordance with the following expression:

Improvement rate (%)=(C4 dicarboxylic acid productivity in mutant/C4 dicarboxylic acid productivity of host or control)×100−100

The mutant herein refers to a cell obtained by modifying a host cell such that a predetermined trait is changed. The host refers to a host of the mutant (parent cell or parent organism). The control refers to a cell or organism which belongs to a different type from the host cell and has been modified in the same manner as in the mutant or refers to a host cell or organism which has been not modified (for example, a host cell or organism to which a vector only or a control sequence was introduced). The improvement rate of C4 dicarboxylic acid productivity in a mutant is preferably calculated based on the C4 dicarboxylic acid productivity of each cell or organism at a time when the C4 dicarboxylic acid-production speed of the mutant reaches a maximum. In the specification, “a mutant having improved C4 dicarboxylic acid productivity by X % or more” refers to a mutant exhibiting an improvement rate of C4 dicarboxylic acid productivity, calculated in accordance with the above expression, of X % or more. The “improvement of C4 dicarboxylic acid productivity by X % or more” in a mutant means that the improvement rate of C4 dicarboxylic acid productivity of the mutant, calculated in accordance with the above expression, is X % or more.

Examples of the C4 dicarboxylic acid to be produced by the present invention include fumaric acid, malic acid and succinic acid, preferably fumaric acid and malic acid, more preferably fumaric acid.

In the specification, “malic enzyme activity” refers to an activity decarboxylating malic acid to produce pyruvic acid and CO₂, and preferably refers to an activity to catalyze a reaction of oxidatively decarboxylating malic acid coupled with reduction of NAD⁺ or NADP⁺ to produce pyruvic acid and CO₂ and NADH or NADPH, as shown below.

L-malate+NAD(P)⁺↔pyruvate+CO₂+NAD(P)H

The malic enzyme activity can be measured by a known method (for example, a method described in W. Tang et al., Mol. Biotechnol., 2010, 45: 121-128).

In the specification, “malic enzyme” (ME) refers to an enzyme having malic enzyme activity as mentioned above. Examples thereof include NAD-malic enzymes and NADP-malic enzymes, which are classified into EC1.1.1.38 (NAD-ME, which can use NAD⁺ alone), EC1.1.1.39 (NAD(P)-ME, which can use both NAD⁺ and NADP⁺) or EC1.1.1.40 (NADP-ME, which can use NADP⁺ alone).

(2. Mutant Filamentous Fungus and Production Method Thereof)

(2.1. Mutant Filamentous Fungus)

In an aspect, the present invention provides a mutant filamentous fungus having enhanced expression of a polypeptide having malic enzyme activity.

In an embodiment, examples of the polypeptide having malic enzyme activity to be enhanced in expression in the mutant filamentous fungus of the present invention include the following polypeptides:
(a) a polypeptide consisting of the amino acid sequence represented by SEQ ID No: 2,
(b) a polypeptide consisting of an amino acid sequence having an identity of at least 90% with the amino acid sequence represented by SEQ ID No: 2 and having malic enzyme activity, and
(c) a polypeptide consisting of an amino acid sequence having deletion, substitution, addition or insertion of one or more amino acids with respect to the amino acid sequence represented by SEQ ID No: 2 and having malic enzyme activity.

The polypeptide consisting of the amino acid sequence represented by SEQ ID No: 2 is malic enzyme derived from Rhizopus. The polypeptide consisting of the amino acid sequence represented by SEQ ID No: 2 is registered as RO3G_04512 and encoded by the gene consisting of the nucleotide sequence represented by SEQ ID No: 1.

As the polypeptide having malic enzyme activity to be enhanced in expression in the mutant filamentous fungus of the present invention, one or more polypeptides selected from the aforementioned group consisting of the polypeptides (a) to (c) can be mentioned.

(2.2. Production of Mutant Filamentous Fungus)

The mutant filamentous fungus of the present invention can be produced by modifying a filamentous fungus and enhancing expression of the polypeptide having malic enzyme activity. Accordingly, in a further aspect, the present invention provides a method for producing a mutant filamentous fungus, comprising enhancing expression of the polypeptide having malic enzyme activity in a host filamentous fungus.

As the host filamentous fungus of the mutant filamentous fungus of the present invention, all filamentous fungi belonging to the subdivision Eumycota and Ooamycota (defined by Hawksworth et al., In, Ainsworth and Bisby's Dictionary of The Fungi, 8th edition, 1995, CAB International, bUniversity, Press, Cambridge, UK) are included.

Preferable examples of the host filamentous fungus of the mutant filamentous fungus of the present invention include filamentous fungi of the genus Acremonium, Aspergillus, Aureobasidium, Bjerkandera, Ceriporiopsis, Chrysosporium, Coprinus, Coriolus, Cryptococcus, Pilibasidium, Fusarium, Humicola, Magnaporthe, Mucor, Myceliophthora, Neocallimastix, Neurospora, Paecilomyces, Parasitella, Penicillium, Phanerochaete, Phiebia, Piromyces, Pleurotus, Rhizopus, Bchizophyllum, Talaromyces, Thermoascus, Thielavia, Tolypocladium, Trametes and Trichoderma. Of these, in view of C4 dicarboxylic acid productivity, filamentous fungi of the genus Rhizopus such as Rhizopus delemar, Rhizopus arrhizus, Rhizopus chinensis, Rhizopus nigricans, Rhizopus tonkinensis, Rhizopus tritici and Rhizopus oryzae are preferable; Rhizopus delemar and Rhizopus oryzae are more preferable; and Rhizopus delemar is further preferable.

In an embodiment, the host filamentous fungus of the mutant filamentous fungus of the present invention may be a mutant strain of Rhizopus. Examples of the mutant strain include an alcohol dehydrogenase gene-deficient (Aadh) strain (Patent Application 2016-000184, which is incorporated in its entirety in the specification as a reference); and a pyruvate decarboxylase gene-deficient (Apdc) strain (PCT/JP2017/003647, which is incorporated in its entirety in the specification as a reference).

As a means for enhancing expression of the polypeptide having malic enzyme activity in a host filamentous fungus, a method of introducing a gene encoding the polypeptide in the host cell extracellularly so as to allow expression or a method of modifying a regulatory region for a gene encoding the polypeptide on the host genome to improve transcription amount of the gene in the host is mentioned.

In a preferable embodiment, expression of the polypeptide having malic enzyme activity according to the present invention is enhanced by introducing a DNA fragment or vector containing a gene encoding the polypeptide into a host filamentous fungus. The expression level of a desired polypeptide having malic enzyme activity is increased by expressing the gene encoding the polypeptide having malic enzyme activity contained in the DNA fragment or vector.

In a preferable embodiment, as the gene encoding the polypeptide having malic enzyme activity to be enhanced in expression, the followings are mentioned:
(a′) a polynucleotide consisting of the nucleotide sequence represented by SEQ ID No: 1,
(b′) a polynucleotide consisting of a nucleotide sequence having an identity of at least 90% with the nucleotide sequence represented by SEQ ID No: 1 and encoding a polypeptide having malic enzyme activity, and
(c′) a polynucleotide consisting of a nucleotide sequence having deletion, substitution, addition or insertion of one or more nucleotides with respect to the nucleotide sequence represented by SEQ ID No: 1 and encoding a polypeptide having malic enzyme activity.

In the method for producing a mutant filamentous fungus according to the present invention, the aforementioned polynucleotides (a′) to (c′) can be used alone or in combination (two or more).

The aforementioned polynucleotides may be in a single stranded or double stranded form; or may be DNA or RNA. The DNA may be cDNA or an artificial DNA such as chemically synthesized DNA.

The polynucleotides (a′) to (c′) can be synthesized in a genetic engineering process or a chemical process. For example, the polynucleotide represented by SEQ ID No: 1 can be prepared by isolating it from Rhizopus such as Rhizopus delemar and Rhizopus oryzae or chemically synthesized based on the nucleotide sequence represented by SEQ ID No: 1. The polynucleotide consisting of a nucleotide sequence having an identity of at least 90% with the nucleotide sequence represented by SEQ ID No: 1 or a polynucleotide consisting of a nucleotide sequence having deletion, substitution, addition or insertion of one or more nucleotides with respect to the nucleotide sequence represented by SEQ ID No: 1 can be produced by introducing a mutation into, for example, a polynucleotide consisting of the nucleotide sequence represented by SEQ ID No: 1 by a known mutagenesis such as UV irradiation and a site-directed mutagenesis.

Examples of a method of introducing a mutation such as deletion, substitution, addition or insertion of a nucleotide(s) in the nucleotide sequence include, for example, mutagenesis with a chemical mutagen such as ethyl methanesulfonate, N-methyl-N-nitrosoguanidine and nitrous acid, or a physical mutagen such as an ultraviolet ray, X ray, gamma ray and an ion beam, a site-directed mutagenesis and a method described by Dieffenbach et al. (Cold Spring Harbar Laboratory Press, New York, 581-621, 1995). Examples of the site-specific mutagenesis method include a method using Splicing overlap extension (SOE) PCR (Horton et al., Gene 77, 61-68, 1989), ODA method (Hashimoto-Gotoh et al., Gene, 152, 271-276, 1995) and Kunkel method (Kunkel, T. A., Proc. Natl. Acad. Sci. USA, 1985, 82, 488). Alternatively, a commercially available kit for site-directed mutagenesis such as Site-Directed Mutagenesis System Mutan-SuperExpress Km kit (Takara Bio Inc.), Transformer™ Site-Directed Mutagenesis kit (Clontech) and KOD-Plus-Mutagenesis Kit (Toyobo Co., Ltd.), can be used.

Preferably, the vector containing the polynucleotide to be introduced into a host filamentous fungus is an expression vector. The vector is preferably an expression vector capable of introducing the polynucleotide of the present invention in a host and expressing the polynucleotide in the host. The vector preferably contains the polynucleotide and a regulatory region operably linked to the polynucleotide. The vector may be a vector capable of extrachromosomally and autonomously proliferating and replicating such as a plasmid, or may be a vector to be incorporated intrachromosomally.

Examples of the vector include pBluescript II SK (−) (Stratagene), a pUC vector such as pUC18, pUC18/19, pUC118/119 (Takara Bio Inc.), a pET vector (Takara Bio Inc.), a pGEX vector (GE healthcare), a pCold vector (Takara Bio Inc.), a pHY300PLK (Takara Bio Inc.), pUB110 (Mckenzie, T. et al., 1986, Plasmid 15 (2): 93-103), pBR322 (Takara Bio Inc.), pRS403 (Stratagene), pMW218/219 (Nippon Gene Co., Ltd.), a pRI vector such as pRI909/910 (Takara Bio Inc.), pPTR1/2 (Takara Bio Inc.), a pBI vector (Clontech), an IN3 vector (Inplanta Innovations Inc.), pDJB2 (D. J. Ballance et al., Gene, 36, 321-331, 1985), pAB4-1 (van Hartingsveldt W et al., Mol Gen Genet, 206, 71-75, 1987), μLeu4 (M. I. G. Roncero et al., Gene, 84, 335-343, 1989), pPyr225 (C. D. Skory et al., Mol Genet Genomics, 268, 397-406, 2002) and pFG1 (Gruber, F. et al., Curr Genet, 18, 447-451, 1990).

As the DNA fragment containing the polynucleotide to be introduced into a host filamentous fungus, for example, a PCR-amplified DNA fragment and a restriction enzyme-cleaved DNA fragment, may be mentioned. Preferably, the DNA fragment may be an expression cassette containing the polynucleotide and a regulatory region operably linked thereto.

The regulatory region to be contained in the vector or the DNA fragment is a sequence for expressing the introduced polynucleotide in a host cell into which the vector or DNA fragment has been introduced and, for example, an expression regulatory region such as a promoter and a terminator, and a replicator are mentioned. The type of the regulatory region can be appropriately selected depending upon the type of host into which a vector or a DNA fragment is introduced. If necessary, the vector or DNA fragment may further have a selection marker such as an antibiotic resistance gene and amino acid synthesis-related genes.

Preferably, the regulatory region contained in the vector or DNA fragment is a regulatory region having a higher transcriptional activity than regulatory regions for the polynucleotides (a′) to (c′) originally present in the host genome (so-called an enhanced regulatory region). Examples of the enhanced regulatory region of Rhizopus include, but are not limited to, ldhA promoter (U.S. Pat. No. 6,268,189), pgk1 promoter (WO-A-2001/73083), pgk2 promoter (WO-A-2001/72967), pdcA promoter and amyA promoter (Archives of Microbiology, 2006, 186: 41-50), tef and 18SrRNA promoter (US-A-2010/112651) and adhi promoter (WO-A-2017/022583) (the documents cited in this paragraph are incorporated in their entirety in the specification as references). Examples of the enhanced regulatory region further include, but are not limited to, a regulatory region of rRNA operon and a regulatory region of a gene encoding a ribosomal protein.

A desired polynucleotide and regulatory region contained in the vector or DNA fragment may be introduced in the nucleus or genome of the host. Alternatively, the desired polynucleotide contained in the vector or DNA fragment may be directly introduced in a host genome and operably linked to a high expression promoter on the genome. As a means for introducing the polynucleotide in a genome, homologous recombination is mentioned.

In introducing the vector or a DNA fragment in the host filamentous fungus, a general transformation method such as an electroporation method, a transformation method, a transfection method, a conjugation method, a protoplast method, a particle-gun method and an agrobacterium method, can be used.

Examples of the means for introducing the vector or DNA fragment in a host genome include, but are not limited to, genome editing using an artificial DNA nucleases or Programmable nuclease. Examples of a technique for the genome editing include TALEN (transcription activator-like effector nuclease), ZFN (zinc-finger nuclease) or CRISPR (Clustered Regularly Interspaced Short Palindromic Repeat)-Cas9 system, CRISPR-Cpf1 and Homing endonuclease and compact designer TALEN. Kits for genome editing based on these techniques are commercially available and can be purchased from, for example, Life technologies, Cellectis, Transposagen Biopharmaceuticals.

A mutant having a desired vector or DNA fragment introduced therein can be selected by using a selection marker. For example, when the selection marker is an antibiotic resistance gene, the mutant having a desired vector or DNA fragment introduced therein can be selected by culturing cells in the medium containing the antibiotic. When the selection marker is, for example, an auxotrophy-related gene such as an amino acid synthesis-related gene and base synthesis-related genes, a gene is transferred into the auxotrophic host and thereafter a mutant having a desired vector or DNA fragment introduced therein can be selected based on the presence or absence of the auxotrophy used as an indicator. Alternatively, introduction of a desired vector or DNA fragment can be confirmed by examining the DNA sequence of the mutant by e.g., PCR.

In another preferable embodiment, expression of a polypeptide having malic enzyme activity according to the present invention is enhanced by modifying the regulatory region of a gene encoding the polypeptide on the genome of a host filamentous fungus to improve the transcription amount of the gene. As the target gene to be improved in transcription amount in the embodiment, any one or more of the polynucleotides (a′) to (c′) are mentioned.

A genome modification procedure for improving the transcription amount of the target gene is, for example, as follows: the aforementioned enhanced regulatory region is substituted for or inserted into the regulatory region of the target gene on the host genome and operably linked to the target gene. As a means for substitution or insertion of a genomic region, homologous recombination is mentioned. In addition, the technique for genome editing mentioned above may be used in combination.

The mutant filamentous fungus of the present invention obtained in the above procedure has improved malic enzyme activity, compared to its host (parent filamentous fungus). The malic enzyme activity of the mutant filamentous fungus of the present invention is preferably 1.1 times or more, more preferably 1.5 times or more, further preferably 2 times or more as large as that of the host.

(2.3. Improvement of C4 Dicarboxylic Acid Productivity)

The mutant filamentous fungus of the present invention has improved C4 dicarboxylic acid productivity, compared to its host. The C4 dicarboxylic acid productivity of the mutant filamentous fungus of the present invention is improved by preferably 10% or more, more preferably 20% or more, further preferably 30% or more as large as that of the host.

(3. Production of C4 Dicarboxylic Acid)

The mutant filamentous fungus of the present invention has improved C4 dicarboxylic acid productivity. Accordingly, in a further aspect, the present invention provides a method for producing a C4 dicarboxylic acid, comprising culturing the mutant filamentous fungus of the present invention. The C4 dicarboxylic acid produced by the production method of the present invention includes fumaric acid, malic acid and succinic acid and the C4 dicarboxylic acid is preferably fumaric acid and malic acid, more preferably fumaric acid.

The medium and culture conditions for culturing the mutant filamentous fungus can be appropriately selected depending upon the type of host for the mutant filamentous fungus. Usually, a medium and culture condition routinely used for a host for the mutant filamentous fungus can be employed.

For example, the culture temperature may be from 10° C. to 50° C., and preferably from 25° C. to 45° C. The culture period, which is not particularly limited as long as it is the period during which a desired C4 dicarboxylic acid is sufficiently produced, may be, for example, from 1 to 240 hours, preferably from 12 to 120 hours and preferably from 24 to 72 hours. Culture is preferably carried out while stirring or under aeration.

As a medium for culturing a filamentous fungus, a medium routinely used may be used. The medium is preferably a liquid medium, and the medium may be any one of a synthetic medium, a natural medium, and a semisynthetic medium obtained by adding a natural component to a synthesis medium. A commercially available medium such as PDB medium (potato dextrose medium, manufactured by e.g., Becton, Dickinson and Company), PDA medium (manufactured by e.g., Becton, Dickinson and Company), LB medium (Luria-Bertani medium, manufactured by e.g., Nihon Pharmaceutical Co., Ltd. (brand name e.g., “DAIGO”)), NB medium (Nutrient Broth, manufactured by e.g., Becton, Dickinson and Company), SB medium (Sabouraud medium, manufactured by e.g., OXOID Ltd.) and SD medium (Synthetic Dropout Broth; for example, Clontech), can be used. The medium usually contains e.g., a carbon source, a nitrogen source and an inorganic salt; however, components and composition of the medium can be appropriately selected.

Now, the composition of a preferable medium for culturing a filamentous fungus will be more specifically described, below. The concentrations of individual components in the medium described below represent initial concentrations thereof (at the preparation of a medium or at the start of culturing).

Examples of the carbon source in the medium as mentioned above include glucose, maltose, starch hydrolysate, fructose, xylose and sucrose. Of them, glucose and fructose are preferable. These sugars can be used alone or in combination of two or more. The concentration of the carbon source in the medium is preferably 1% (w/v) or more, more preferably 5% (w/v) or more, further preferably 7.5% (w/v) or more; and preferably 40% (w/v) or less and more preferably 30% (w/v) or less. Alternatively, the concentration of the carbon source in the medium is preferably from 1 to 40% (w/v), more preferably from 5 to 30% (w/v), further preferably from 7.5 to 30% (w/v).

Examples of the nitrogen source in the medium include a nitrogen-containing compound such as ammonium sulfate, urea, ammonium nitrate, potassium nitrate and sodium nitrate. The concentration of the nitrogen source in the medium is preferably from 0.001 to 0.5% (w/v), more preferably from 0.001 to 0.2% (w/v).

The medium can contain e.g., a sulfate, a magnesium salt and a zinc salt. Examples of the sulfate include magnesium sulfate, zinc sulfate, potassium sulfate, sodium sulfate and ammonium sulfate. Examples of the magnesium salt include magnesium sulfate, magnesium nitrate and magnesium chloride. Examples of the zinc salt include zinc sulfate, zinc nitrate and zinc chloride. The concentration of the sulfate in a medium is preferably from 0.001 to 0.5% (w/v) and more preferably from 0.001 to 0.2% (w/v). The concentration of the magnesium salt in the medium is preferably from 0.001 to 0.5% (w/v), more preferably from 0.01 to 0.1% (w/v). The concentration of the zinc salt in the medium is preferably from 0.001 to 0.05% (w/v), more preferably from 0.005 to 0.05% (w/v).

The pH (25° C.) of the medium is preferably from 3 to 7, more preferably from 3.5 to 6. The pH of a medium can be adjusted with a base such as calcium hydroxide, sodium hydroxide, calcium carbonate and ammonia or an acid such as a sulfuric acid and hydrochloric acid.

A preferable example of the medium includes a liquid medium containing from 7.5 to 30% of carbon source, from 0.001 to 0.2% of ammonium sulfate, from 0.01 to 0.6% of potassium dihydrogen phosphate, from 0.01 to 0.1% magnesium sulfate heptahydrate, from 0.005 to 0.05% of zinc sulfate heptahydrate and from 3.75 to 20% calcium carbonate (concentrations are all expressed by % (w/v)).

To efficiently produce a C4 dicarboxylic acid using a filamentous fungus, production may be carried out in the steps mentioned below. More specifically, a C4 dicarboxylic acid can be efficiently produced by preparing a spore suspension of a filamentous fungus (step A), culturing the spore suspension in a culture solution to germinate the spores, thereby preparing a mycelia (step B1), preferably further proliferating the mycelia (step B2), and then culturing the mycelia prepared to produce the C4 dicarboxylic acid (step C). Note that, the step of culturing the mutant filamentous fungus in the present invention is not limited to the following steps.

### <Step A: Preparation of Spore Suspension>

Spores of a mutant filamentous fungus are inoculated, for example, into a medium such as an inorganic agar medium (composition example: 2% glucose, 0.1% ammonium sulfate, 0.06% potassium dihydrogen phosphate, 0.025% magnesium sulfate heptahydrate, 0.009% zinc sulfate heptahydrate and 1.5% agar (concentrations are all expressed by % (w/v))) and PDA medium, and subjected to stationary culture at from 10 to 40° C., preferably from 27 to 30° C. for from 7 to 10 days to form spores, which are then suspended in e.g., physiological saline to prepare a spore suspension. The spore suspension may or may not contain mycelia.

### <Step B1: Preparation of Mycelia>

The spore suspension obtained in step A is inoculated in a culture solution and cultured to germinate spores, thereby obtaining mycelia. The number of spores of a filamentous fungus to be inoculated in the culture solution is from 1×10² to 1×10⁸ spores/mL (culture solution), preferably from 1×10² to 5×10⁴ spores/mL (culture solution), more preferably from 5×10² to 1×10⁴ spores/mL (culture solution), further preferably from 1×10³ to 1×10⁴ spores/mL (culture solution). As the culture solution, a commercially available medium such as PDB medium, LB medium, NB medium, SB medium and SD medium, can be used. In view of germinating rate and mycelial growth, a carbon source including a monosaccharide such as glucose and xylose, an oligosaccharide such as sucrose, lactose and maltose, or a polysaccharide such as starch; a biological substance such as glycerin and citric acid; a nitrogen source such as ammonium sulfate, urea and amino acid; and other inorganic substances such as various salts including a sodium salt, a potassium salt, a magnesium salt, a zinc salt, an iron salt and a phosphate may be appropriately added to the culture solution. The preferable concentrations of a monosaccharide, oligosaccharide, polysaccharide and glycerin are from 0.1 to 30% (w/v); the preferable concentration of citric acid is from 0.01 to 10% (w/v); the preferable concentrations of ammonium sulfate, urea and amino acid are from 0.01 to 1% (w/v); and the preferable concentration of an inorganic substance is from 0.0001 to 0.5% (w/v). To the culture solution, the spore suspension is inoculated. The obtained solution is cultured for preferably from 24 to 120 hours and more preferably from 48 to 72 hours, while stirring at preferably from 80 to 250 rpm and more preferably from 100 to 170 rpm and controlling a culture temperature to be from 25 to 42.5° C. The amount of the culture solution to be subjected to culture, which may be appropriately controlled depending upon the size of the culture vessel; may be, about from 50 to 100 mL, in the case where the culture vessel is e.g., a 200 mL flask with a baffle, and about from 100 to 300 mL in the case where the culture vessel is a 500 mL flask with a baffle. Owing to the culture, the spores inoculated are germinated and grow into mycelia.

### <Step B2: Proliferation of Mycelia>

In view of improvement of C4 dicarboxylic acid productivity, it is preferable to perform a step (step B2) of further culturing the mycelia obtained in step B1 to proliferate. The culture solution for proliferation used in step B2 is not particularly limited and may be an inorganic culture solution routinely used and containing glucose. For example, a culture solution containing from 7.5 to 30% of glucose, from 0.001 to 0.2% of ammonium sulfate, from 0.01 to 0.6% of potassium dihydrogen phosphate, from 0.01 to 0.1% of magnesium sulfate heptahydrate, from 0.00.5 to 0.05% of zinc sulfate heptahydrate and from 3.75 to 20% of calcium carbonate (concentrations are all expressed by % (w/v)), may be mentioned. The amount of culture solution may be appropriately controlled depending upon the size of a culture vessel. For example, in the case where the culture vessel is a 500 mL Erlenmeyer flask, the amount of culture solution may be from 50 to 300 mL, preferably from 100 to 200 mL. To the culture solution, the mycelia cultured in step B1 were inoculated so as to obtain a rate of, as wet weight, from 1 to 6 g of mycelia/100 mL (culture solution), preferably from 3 to 4 g of mycelia/100 mL (culture solution), and the obtained solution is cultured for from 12 to 120 hours, preferably from 24 to 72 hours while stirring at from 100 to 300 rpm, preferably from 170 to 230 rpm and controlling a culture temperature to be from 25 to 42.5° C.

### <Step C: Production of a C4 Dicarboxylic Acid>

The mycelia of a filamentous fungus obtained in the aforementioned procedure (step B1 or B2) are cultured to allow the fungus to produce a C4 dicarboxylic acid. The conditions of the cultivation may follow the culture conditions mentioned above and routinely used for filamentous fungi. The amount of medium may be about from 20 to 80 mL in the case of a 200 mL Erlenmeyer flask, about from 50 to 200 mL in the case of a 500 mL Erlenmeyer flask and about from 10 L to 15 L in the case of a 30 L jar fermenter; however, the amount of medium may be appropriately controlled depending upon the size of the culture vessel. The inoculation amount of the mycelia obtained in step B1 or B2 to the medium may be preferably, as wet weight, from 5 g to 90 g of mycelia/100 mL (medium), more preferably from 5 g to 50 g of mycelia/100 mL (medium). Culture is preferably performed at a temperature of from 25 to 45° C. for from 2 hours to 240 hours, preferably from 12 hours to 120 hours while stirring at from 100 to 300 rpm, preferably from 150 to 230 rpm. If a jar fermenter is used, aeration is preferably performed at from 0.05 to 2 vvm, more preferably from 0.1 to 1.5 vvm.

The mutant filamentous fungus of the present invention is cultured in the above procedure to produce a C4 dicarboxylic acid. After cultivation, the C4 dicarboxylic acid is collected from the cultured broth. If necessary, the C4 dicarboxylic acid collected may be further purified. A method for collecting or purifying a C4 dicarboxylic acid from the cultured broth is not particularly limited and may be performed in accordance with a collection or purification method known in the art. For example, the C4 dicarboxylic acid in the cultured broth can be collected or purified by removing cells and the like from the cultured broth by a method such as a gradient method, filtration and centrifugation, if necessary concentrating the remaining cultured broth, and then subjecting the concentrate to a method such as a crystallization method, an ion exchange method and a solvent extraction method or a combination of these.

The mutant filamentous fungus of the present invention separated from the cultured broth can be reused in producing a C4 dicarboxylic acid. For example, to the mutant filamentous fungus of the present invention separated from the cultured broth, the medium as mentioned above is newly added. The mixture is cultured again under the aforementioned conditions to produce a C4 dicarboxylic acid. Thei the C4 dicarboxylic acid produced can be collected from the medium. This process can be further repeated. In the production method of the present invention, culture of a mutant filamentous fungus and collection of a C4 dicarboxylic acid can be performed in either one of a batch, semi-batch and continuous mode.

### 4. Illustrative Embodiments

As an illustrative embodiment of the present invention, the following substances, production method, use and method will be further disclosed herein. However, the present invention is not limited to these embodiments.

[1] A mutant filamentous fungus having enhanced expression of at least one polypeptide selected from the group consisting of the followings:
(a) a polypeptide consisting of the amino acid sequence represented by SEQ ID No: 2,
(b) a polypeptide consisting of an amino acid sequence having an identity of at least 90% with the amino acid sequence represented by SEQ ID No: 2 and having malic enzyme activity, and
(c) a polypeptide consisting of an amino acid sequence having deletion, substitution, addition or insertion of one or more amino acids with respect to the amino acid sequence represented by SEQ ID No: 2 and having malic enzyme activity.

[2] Preferably, the mutant filamentous fungus according to [1], comprising a DNA fragment or vector introduced therein, wherein the DNA fragment or vector comprises at least one polynucleotide selected from the group consisting of the followings:
(a′) a polynucleotide consisting of the nucleotide sequence represented by SEQ ID No: 1,
(b′) a polynucleotide consisting of a nucleotide sequence having an identity of at least 90% with the nucleotide sequence represented by SEQ ID No: 1 and encoding a polypeptide having malic enzyme activity, and
(c′) a polynucleotide consisting of a nucleotide sequence having deletion, substitution, addition or insertion of one or more nucleotides with respect to the nucleotide sequence represented by SEQ ID No: 1 and encoding a polypeptide having malic enzyme activity.

[3] Preferably, the mutant filamentous fungus according to [2], in which the DNA fragment or vector further comprises a regulatory region operably linked to the polynucleotide.

[4] Preferably, the mutant filamentous fungus according to [2] or [3], in which the DNA fragment or vector is introduced in a nucleus or genome.

[5] Preferably, the mutant filamentous fungus according to [1], in which a regulatory region for a gene encoding the polypeptide on the genome is modified to improve the transcription amount of the gene and the gene is at least one selected from the group consisting of the followings:
(a′) a polynucleotide consisting of the nucleotide sequence represented by SEQ ID No: 1,
(b′) a polynucleotide consisting of a nucleotide sequence having an identity of at least 90% with the nucleotide sequence represented by SEQ ID No: 1 and encoding a polypeptide having malic enzyme activity, and
(c′) a polynucleotide consisting of a nucleotide sequence having deletion, substitution, addition or insertion of one or more nucleotides with respect to the nucleotide sequence represented by SEQ ID No: 1 and encoding a polypeptide having malic enzyme activity.

[6] Preferably, the mutant filamentous fungus according to [5], in which an enhanced regulatory region is substituted for or inserted into the regulatory region.

[7] Preferably, the mutant filamentous fungus according to any one of [1] to [6], in which the filamentous fungus is Rhizopus.

[8] The mutant filamentous fungus according to [7], in which
the Rhizopus is
preferably Rhizopus delemer or Rhizopus orise,
more preferably Rhizopus delemer.

[9] Preferably, the mutant filamentous fungus according to [7] or [8], in which the Rhizopus is a Δadh or Δpdc strain.

[10] The mutant filamentous fungus according to any one of [1] to [9], in which the C4 dicarboxylic acid productivity is improved by preferably 10% or more, more preferably 20% or more, further preferably 30% or more.

[11] The mutant filamentous fungus according to [10], in which the C4 dicarboxylic acid is
preferably fumaric acid, malic acid or succinic acid,
more preferably fumaric acid or malic acid,
further preferably fumaric acid.

[12] A production method for a C4 dicarboxylic acid, comprising culturing the mutant filamentous fungus according to any one of [1] to [11].

[13] The production method according to [12], further comprising collecting the C4 dicarboxylic acid from the cultured broth.

[14] The production method according to [12] or [13], in which the C4 dicarboxylic acid is
preferably fumaric acid, malic acid or succinic acid,
more preferably fumaric acid or malic acid,
further preferably fumaric acid.

[15] A method for producing a mutant filamentous fungus, comprising enhancing, in a host filamentous fungus, expression of at least one polypeptide selected from the group consisting of the followings:
(a) a polypeptide consisting of the amino acid sequence represented by SEQ ID No: 2,
(b) a polypeptide consisting of an amino acid sequence having an identity of at least 90% with the amino acid sequence represented by SEQ ID No: 2 and having malic enzyme activity, and
(c) a polypeptide consisting of an amino acid sequence having deletion, substitution, addition or insertion of one or more amino acids with respect to the amino acid sequence represented by SEQ ID No: 2 and having malic enzyme activity.

[16] A method for improving C4 dicarboxylic acid productivity in a filamentous fungus, comprising: enhancing, in the filamentous fungus, expression of at least one polypeptide selected from the group consisting of the followings:
(a) a polypeptide consisting of the amino acid sequence represented by SEQ ID No: 2,
(b) a polypeptide consisting of an amino acid sequence having an identity of at least 90% with the amino acid sequence represented by SEQ ID No: 2 and having malic enzyme activity, and
(c) a polypeptide consisting of an amino acid sequence having deletion, substitution, addition or insertion of one or more amino acids with respect to the amino acid sequence represented by SEQ ID No: 2 and having malic enzyme activity.

[17] Preferably, the method according to [15] or [16], in which the enhancing expression comprises introducing a DNA fragment or vector, wherein the DNA fragment or vector comprises at least one polynucleotide selected from the group consisting of the followings:
(a′) a polynucleotide consisting of the nucleotide sequence represented by SEQ ID No: 1,
(b′) a polynucleotide consisting of a nucleotide sequence having an identity of at least 90% with the nucleotide sequence represented by SEQ ID No: 1 and encoding a polypeptide having malic enzyme activity, and
(c′) a polynucleotide consisting of a nucleotide sequence having deletion, substitution, addition or insertion of one or more nucleotides with respect to the nucleotide sequence represented by SEQ ID No: 1 and encoding a polypeptide having malic enzyme activity.

[18] Preferably, the method according to [17], in which the DNA fragment or vector further comprises a regulatory region operably linked to the polynucleotide.

[19] Preferably, the method according to [17] or [18], in which the introducing a DNA fragment or vector comprises introducing the DNA fragment or vector into a nucleus or genome.

[20] Preferably, the method according to [15] or [16], comprising modifying a regulatory region for a gene encoding the polypeptide on the genome to improve a transcription amount of the gene, and the gene is at least one selected from the group consisting of the followings:
(a′) a polynucleotide consisting of the nucleotide sequence represented by SEQ ID No: 1,
(b′) a polynucleotide consisting of a nucleotide sequence having an identity of at least 90% with the nucleotide sequence represented by SEQ ID No: 1 and encoding a polypeptide having malic enzyme activity, and
(c′) a polynucleotide consisting of a nucleotide sequence having deletion, substitution, addition or insertion of one or more nucleotides with respect to the nucleotide sequence represented by SEQ ID No: 1 and encoding a polypeptide having malic enzyme activity.

[21] Preferably, the method according to [20], in which modifying the regulatory region includes substituting or inserting an enhanced regulatory region for or into the regulatory region.

[22] Preferably, the method according to any one of [15] to [21], in which the filamentous fungus is Rhizopus.

[23] The method according to [22], in which the Rhizopus is
preferably, Rhizopus delemer or Rhizopus orise,
more preferably Rhizopus delemer.

[24] Preferably, the method according to [22] or [23], in which the Rhizopus is a Δadh or Δpdc strain.

[25] The method according to any one of [15] to [24], in which the C4 dicarboxylic acid productivity of the filamentous fungus having enhanced expression of the polypeptide is improved by preferably 10% or more, more preferably 20% or more, further preferably 30% or more.

[26] The method according to [25], in which the C4 dicarboxylic acid is
preferably fumaric acid, malic acid or succinic acid,
more preferably fumaric acid or malic acid,
further preferably fumaric acid.

### EXAMPLES

The present invention will be more specifically described below based on Examples; however, the present invention is not limited to these.

### Example 1 Production of Mutant Filamentous Fungus

The PCR primers used in this Example are shown in Table 1-1 and Table 1-2.

TABLE 1-1

| | | SEQ |
|---|---|---|
| | | ID |
| Primer | Sequence (5′→3′) | No. |
| oJK162 | cgagctcgaattatttaaatgaacagcaagttaataatctagaggg | 12 |
| oJK163 | tatgaccatgattacgatgagaggcaaaatgaagcgtac | 13 |
| oJK164 | atttaaataattcgagctcggtacccgggg | 14 |
| oJK165 | cgtaatcatggtcatagctg | 15 |
| oJK202 | tagagggaaaaagagagaattgaaatagg | 16 |
| oJK204 | ttttgttatttaattgtattaattgataatg | 17 |
| oJK205 | aattaaataacaaaatcattttaattacgcattttc | 18 |
| oJK216 | catgattacgcggccgcgccattataatgcactagtg | 19 |
| oJK210 | ctctttttccctctaatgagaggcaaaatgaagcgtac | 20 |
| oJK211 | aattaaataacaaaaatgtcttctatcgaaacctccaaaatctc | 21 |
| trpC-Iost-F | tttaaattagagggaaaaagagagaattgaaatag | 22 |
| trpC-Iost-R | tccctctaatttaaatgaattcgagctcggtaccc | 23 |
| NK-141 | aattaaataacaaaaatgttatattcaaaagcatttc | 24 |
| NK-163 | gcgtaattaaaatgactaatgcatgttgggaac | 25 |
| NK-011 | ttttgttatttaattgtattaattg | 26 |
| NK-012 | tcattttaattacgcattttc | 27 |
| pUC18-Pae1-F3 | ctgcaggtcgactctagaggatccccgggtaccg | 28 |
| pUC18-Hind3-R3 | gcttggcactggccgtcgttttacaacgtcgtgac | 29 |
| PDC1-upstr-F | cggccagtgccaagcgcagacttcaacagttggcttttttaagta | 30 |
| PDC1-upstr-R | cattttgcctctcatgtttttaaatttgttttgtagagtattgaata | 31 |
| trpCpro-R | gaacagcaagttaataatctagagggcgc | 32 |
| trpCter-F | atgagaggcaaaatgaagcgtacaaagag | 33 |

TABLE 1-2

| | | SEQ |
|---|---|---|
| | | ID |
| Primer | Sequence (5′→3′) | No. |
| PDC1-downstr-F | attaacttgctgttcaatcttagaattcattttttttttgtatcattcg | 34 |
| PDC1-downstr-R | agagtcgacctgcaggcgtcaataagagcttgaaggttggtgccggatc | 35 |
| oJK899 | gaacagcaagttaataatctagagggcgc | 36 |
| oJK900 | aatcttagaattcattttttttttgtatc | 37 |
| oJK901 | attaacttgctgttctagagggaaaaagagagaattgaaatagg | 38 |
| NK-195 | atgaattctaagattctaatgcatgttgggaac | 39 |
| oJK902 | gcagacttcaacagttggatttttaagta | 40 |
| oJK903 | (p)-gcgtcaataagagcttgaaggttggtgccggatc | 41 |
| PDC1-upstr-F2 | gcagacttcaacagttggcttttttaagta | 42 |
| PDC1-downstr-R-P | (p)-gcgtcaataagagettgaaggttggtgccggatc | 43 |
| adhpro-R | ttttgttatttaattgtattaattgataatg | 44 |
| adhter-F | tcattttaattacgcattttcatttac | 45 |
| adhpro-TALEN-F | aattaaataacaaaaatggactacaaagaccatgacggtg | 46 |
| TAELN-adhter-R | gcgtaattaaaatgattaaaagtttatctcgccgttatta | 47 |
| adhpro-exo1-F | aattaaataacaaaaatgaaaatccaagttgcttctcctattgac | 48 |
| exo1-adhter-R | gcgtaattaaaatgattatcttctttcatgagaaacactaaacttg | 49 |
| NK-069 | cagaaaaacggctgattttagacc | 50 |
| NK-118 | ctgtagtaatgaagtatagaacgaatg | 51 |

(p)Phosphorylated at the 5′ end

### (1) Genome Extraction

To PDA medium, spores of Rhizopus delemar JCM4 (Japan Collection of Microorganisms/Rikcen) 5557 strain (hereinafter referred to as 5557 strain) were inoculated and cultured at 30° C. for 5 days. After completion of the culture, the obtained mycelia were placed together with metal cones for a 3 mL tube (Yasui Kikai Corporation) in a 3 mL homogenizing tube and immediately frozen in liquid nitrogen for 10 minutes or more. Thereafter, the mycelia were homogenized using a multi bead shocker (Yasui Kikai Corporation) at 1,700 rpm for 10 seconds. After completion of the homogenizing, 400 μL of TE Buffer (pH8.0) (Nippon Gene Co., Ltd.) was added to the container and mixed by inversion, and then, 250 μL of an aliquot was transferred to a 1.5 mL tube. From the mycelial solution, a genome was extracted using “Dr. GenTLE (for yeast)” (Takara Bio Inc.) in accordance with the protocol. To 50 μL of the resultant genome solution, 1 μL of RNaseA (Roche) was added and allowed to react at 37° C. for one hour. After completion of the reaction, an equivalent amount of phenol/chloroform was added and mixed by tapping. Thereafter, the mixture was centrifuged at 4° C. and 14,500 rpm for 5 minutes. The supernatant was transferred to a new 1.5 mL tube. The treatment with phenol/chloroform was repeated again and then precipitation with ethanol was performed to obtain a purified genome solution of 5557 strain.

(2) Preparation of cDNA

(i) Extraction of Total RNA

Into 40 mL of liquid medium (0.1 g/L (NH₄)₂SO₄, 0.6 g/L KH₂PO₄, 0.25 g/L MgSO₄.7H₂O, 0.09 g/L ZnSO₄.7H₂O, 50 g/L calcium carbonate, 100 g/L glucose), 6 g, in wet weight, of mycelia of 5557 strain was inoculated and cultured at 35° C. and 170 rpm for 8 hours. Mycelia were collected from the culture solution by filtration and washed twice with 100 mL of 0.85% physiological saline. After completion of the washing, extra water was removed by suction filtration. Then, the mycelia (0.3 g) were weighed out, placed in a 3 mL homogenizing tube together with metal cones for a 3 mL tube (Yasui Kikai Corporation) and immediately placed in liquid nitrogen to freeze. The frozen mycelia thus obtained were homogenized by a multi bead shocker (Yasui Kikai Corporation) at 1,700 rpm for 10 seconds. To the mycelia homogenized, 500 μL of RLT buffer was added and mixed by inversion, and then, 450 μL of an aliquot was subjected to RNeasy Plant Mini Kit (Qiagen) to extract total RNA. To 40 μL of the RNA solution thus obtained, 1 μL of DNaseI (TaKaRa) and 5 μL of 10×DNaseI buffer (USB Corporation) were added. The obtained solution was filled up to 50 μL with RNase free water and allowed to react at 37° C. for 30 minutes or more to remove residual DNA in the solution. DNaseI (1 μL) was further add to the solution, which was allowed to react at 37° C. for 30 minutes and then subjected to phenol/chloroform extraction, followed by ethanol precipitation. The precipitate was dissolved in 50 μL of sterilized water. The concentration and purity of the RNA solution were measured by Qubit (Life Technologies). The RNA solution was appropriately diluted and the RNA extracted was assayed by using Agilent 2100 Bioanalyzer (Agilent) and RNA6000 Pico Kit (Agilent). The resultant RNA solution, which was confirmed to have an RNA degradation index: “RNA Integrity Number (RIN value)” of 6.0 or more, was used as total RNA.

(ii) Synthesis of cDNA

cDNA was synthesized using SuperScriptIII First-Strand Synthesis SuperMix for qRT-PCR (Invitrogen). More specifically, 1 μg of the RNA solution obtained in (i) was filled up to 8 μL with DEPC water. After that, to the RNA solution, 10 μL of 2×RT Reaxtion Mix, and 2 μL of RT Enzyme Mix were added. The mixture was gently mixed and allowed to react at 25° C. for 10 minutes, 50° C. for 30 minutes and 85° C. for 5 minutes. After completion of the reaction, 1 μL of RNaseH was added to the solution, the mixture solution was allowed to react at 37° C. for 20 minutes and the resultant was used as a cDNA solution.

(3) Preparation of Malic Enzyme RdME1 Gene-Containing Plasmid Vector

(i) Introduction of trpC Gene Region in pUC18

Using the genomic DNA of 5557 strain obtained in (1) in the above as a template, a DNA fragment containing a trpC gene (SEQ ID No: 3) was synthesized by PCR using primers oJK162 (SEQ ID No: 12) and oJK163 (SEQ ID No: 13). Subsequently, using plasmid pUC18 as a template, a DNA fragment was amplified by PCR using primers oJK164 (SEQ ID No: 14) and oJK165 (SEQ ID No: 15). The two fragments obtained above were ligated by In-Fusion HD Cloning Kit (Clontech) to construct plasmid pUC18-trpC.

(ii) Cloning of ADH1 Promoter and Terminator

Using the genomic DNA of 5557 strain obtained in (1) in the above as a template, a DNA fragment containing a ADH1 promoter sequence (SEQ ID No: 4) and a DNA fragment containing a terminator sequence (SEQ ID No: 5) were amplified by PCR using a primer pair of oJK202 (SEQ ID No: 16) and oJK204 (SEQ ID No: 17) and a primer pair of oJK205 (SEQ ID No: 18) and oJK216 (SEQ ID No: 19), respectively. Subsequently, using plasmid pUC18-trpC obtained in (i) as a template, a DNA fragment was amplified by PCR using primer oJK210 (SEQ ID No: 20) and oJK211 (SEQ ID No: 21). The three fragments obtained above were ligated in the same manner as (i) to construct plasmid pUC18-trpC-Padh-Tadh. The plasmid thus obtained has ADH1 promoter and terminator arranged downstream of the trpC gene region in order. Further, a Not I restriction enzyme recognition sequence was arranged downstream of ADH1 terminator.

Further, a plasmid vector, which is obtained by removing the trpC gene region from pUC18-trpC-Padh-Tadh, was prepared. More specifically, DNA fragment was amplified by PCR using pUC18-trpC-Padh-Tadh constructed in the above as a template and primers trpC-lost-F (SEQ ID No: 22) and trpC-lost-R (SEQ ID No: 23). This fragment was ligated in the same procedure as in (i) to construct plasmid pUC18-Padh-Tadh.

(iii) Preparation of Plasmid Vector

A gene encoding malic enzyme (hereinafter referred to as RdME1, SEQ ID No: 1) was amplified by PCR using primers NK-141 (SEQ ID No: 24) and NK-163 (SEQ ID No: 25) from the cDNA library prepared in (2). Subsequently, using the plasmid pUC18-trpC-Padh-Tadh obtained in (ii) as a template, a DNA fragment was amplified by PCR using primers NK-011 (SEQ ID No: 26) and NK-012 (SEQ ID No: 27). The two fragments obtained above were ligated in the same procedure as in (i) to construct plasmid pUC18-trpC-Padh-RdME1-Tadh. The plasmid thus obtained has an RdME1 gene inserted between the ADH promoter and the terminator.

(4) Preparation of Plasmid for trpC Knock-in

Plasmid ptrpC-knock-in for knocking-in the trpC gene region was prepared by removing ORF of pdc1 gene at the pdc1 genetic locus. More specifically, plasmid ptrpC-knock-in was constructed by ligating a pUC18 vector fragment amplified by using pUC18 as a template and primers pUC18-Pae1-F3 (SEQ ID No: 28) and pUC18-Hind3-R3 (SEQ ID No: 29); a promoter-site fragment of pdc gene amplified by using the genome of JCM5557 strain as a template and primers PDC1-upstr-F (SEQ ID No: 30) and PDC1-upstr-R (SEQ ID No: 31); a trpC gene-region fragment amplified by using the genome of JCM5557 strain as a template and primers trpCpro-R (SEQ ID No: 32) and trpCter-F (SEQ ID No: 33); and a terminator site fragment of pdc gene amplified by using the genome of JCM5557 strain as a template and primers PDC1-downstr-F (SEQ ID No: 34) and PDC1-downstr-R (SEQ ID No: 35), by means of In-Fusion HD Cloning Kit (Clontech).

(5) Preparation of Construct for Introducing RdME1 Gene

(i) Ligation to Plasmid for trpC Knock-in

A DNA fragment was amplified by PCR using the plasmid ptrpC-knock-in prepared in (4) as a template and primers oJK899 (SEQ ID No: 36) and oJK900 (SEQ ID No: 37).

Subsequently, a DNA fragment was amplified by PCR using plasmid pUC18-trpC-Padh-RdMB1-Tadh obtained in (3) (iii) as a template and primers oJK901 (SEQ ID No: 38) and NK-195 (SEQ ID No: 39). The two fragments obtained above were ligated in the same procedure as in (i) to construct plasmid pUC18-Ppdc-trpC-Padh-RdME1-Tpdc. The plasmid thus obtained contains a sequence for trpC knock-in and RdME1 gene represented by SEQ ID No: 1 and inserted between ADH promoter and PDC terminator.

(ii) Preparation of Single Stranded DNA

A DNA fragment was amplified by PCR using plasmid pUC18-Ppdc-trpC-Padh-RdME1-Tpdc prepared in (i) as a template and primers oJK902 (SEQ ID No: 40) and oJK903 (SEQ ID No: 41, the 5′ end is phosphorylated). Also, a DNA fragment was amplified by PCR using plasmid ptrpC-knock-in as a template and primers PDC1-upstr-F2 (SEQ ID No: 42) and PDC1-downstr-R-P (SEQ ID No: 43, the 5′ end is phosphorylated). After the templates were decomposed by treatment with Dpnl (TOYOBO), the product was purified with a phenol/chloroform/isoamyl alcohol treatment and an ethanol precipitation treatment. The purified product was further treated with Lambda Exonuclease (NEW ENGLAND BioLabs) and purified in the same manner as above to obtain a single stranded DNA. The treatment with Lambda Exonuclease was carried out at 37° C., overnight.

(6) Preparation of TALEN for Pdc1 Gene Disruption

(i) Preparation of TALEN Expression Vector

Custom XTN TALEN (trade name of TALEN provided by Transposagen Biopharmaceuticals) was prepared by outsourcing it to Transposagen Biopharmaceuticals. This is a TALEN kit for targeting a gene encoding pyruvate decarboxylase (PDC) (pdc gene; SEQ ID No: 6) and contains two polynucleotides: LeftTALEN-pdc (SEQ ID No: 7) and RightTALEN-pdc (SEQ ID No: 8), which bind to a region containing pdc gene (SEQ ID No: 9). LeftTALEN-pdc encodes TALEN targeting the sequence: 5′-TGCCTGCTATTAAAATCG-3′ (SEQ ID No: 10) present in the sense strand of pdc gene; whereas, RightTALEN-pdc encodes TALEN targeting the sequence: 5′-TTGATTTCCTTAAGACGG-3′ (SEQ ID No: 11) present in the antisense strand thereof.

A vector expressing TALEN under control of adh promoter and adh terminator was prepared by inserting the polynucleotide encoding LeftTALEN-pdc into the expression vector, pUC18-Padh-Tadh, prepared in the above (3). More specifically, a vector fragment was amplified by PCR using pUC18-Padh-Tadh as a template and primers adhpro-R (SEQ ID No: 44) and adhter-F (SEQ ID No: 45). Subsequently, LeftTALEN-pdc fragment was amplified by PCR using LeftTALEN-pdc as a template and primers adhpro-TALEN-F (SEQ ID No: 46) and TALEN-adhter-R (SEQ ID No: 47). The above two fragments have regions overlapping with each other by 15 bases. These two fragments were ligated by In-Fusion HD cloning kit (Clontech) to obtain a vector containing LeftTALEN-pdc, padh-LeftTALEN-pdc.

Similarly, vector padh-RightTALEN-pdc expressing TALEN under control of adh promoter and adh terminator was prepared by inserting the polynucleotide encoding RightTALEN-pdc into pUC18-Padh-Tadh. For amplification of pUC18-Padh-Tadh fragment, primers adhpro-R (SEQ ID No: 44) and adhter-F (SEQ ID No: 45) were used. For amplification of RightTALEN-pdc fragment, primers adhpro-TALEN-F (SEQ ID No: 46) and TALEN-adhter-R (SEQ ID No: 47) were used.

(ii) Preparation of Exonuclease Expression Vector

Since disruption of target DNA with TALEN is accelerated by expressing exonuclease intracellularly together with TALEN (Scientific Reports, 2013, 3: 1253, DOI: 10. 1038/srep01253, Nat Methods, 2012, 9: 973-975), an exonuclease expression vector, which was to be inserted together with a TALEN expression vector, was prepared.

An exonuclease gene fragment was amplified by using a purified genomic solution of Rhizopus oryzae NRBC5384 strain as a template and primers adhpro-exo1-F (SEQ ID No: 48) and exo1-adhter-R (SEQ ID No: 49). A vector fragment was amplified by PCR using pUC18-Padh-Tadh as a template and primers adhpro-R (SEQ ID No: 44) and adhter-F (SEQ ID No: 45). The above two fragments amplified were ligated by means of In-Fusion HD cloning kit (Clontech) to prepare plasmid padh-exo1.

(7) Introduction of Gene in Host Cell

(i) Preparation of Tryptophan Auxotrophic Strain

A tryptophan auxotrophic strain used as a host cell for gene introduction was screened from strains which had been mutated by ion beam irradiation to the 5557 strain. Ion beam irradiation was carried out at the ion irradiation facility of Takasaki Advanced Radiation Research Institute (TIARA: Takasaki Ion Accelerators for Advanced Radiation Application) of the Japan Atomic Energy Agency. The strain was irradiated with 100 to 1,250 Gray of rays using ¹²C⁵⁺ accelerated by AVP cyclotron at an energy of 220 MeV. Spores were collected from the fungal cells irradiated. From the spores, a tryptophan auxotrophic strain, Rhizopus delemar 02T6 strain (hereinafter, referred to as 02T6 strain) was obtained. The 02T6 Strain has deletion of a single base at position 2093 of the trpC gene coding region (SEQ ID No: 3) (full length: 2,298 bp).

(ii) Preparation of DNA-Gold Particle Mixed Solution

To 100 μL of a gold particle solution (60 mg/mL, INBIO GOLD, particle size 1 μm), 10 μL of a DNA solution (1 μg/μL), which was a mixture of padh-LeftTALEN-pdc, padh-RightTALEN-pdc, and padh-exo1 prepared in the above (6) and the single stranded DNA prepared in the above (5), was added. To this mixture, 40 μL of 0.1 M spermidine was further added and the mixture was sufficiently stirred by vortex. To this, 100 μL of 2.5 M CaCl₂ was added and the mixture was stirred for one minute by vortex and then centrifuged at 6000 rpm for 30 seconds to remove a supernatant. To the resultant precipitate, 200 μL of 70% EtOH was added. The resultant was stirred for 30 seconds by a vortex and centrifuged at 6,000 rpm for 30 seconds and the supernatant was removed therefrom. The resultant precipitate was resuspended in 100 μL of 100% EtOH.

(iii) Gene Introduction

Subsequently, into the spores of 02T6 strain prepared in (i), a gene was introduced by using GDS-80 (Nepa Gene Co., Ltd.) and using the DNA-gold particle solution prepared in (ii). The spores having the gene introduced therein were subjected to stationary culture in an inorganic agar medium (20 g/L glucose, 1 g/L ammonium sulfate, 0.6 g/L potassium dihydrogen phosphate, 0.25 g/L magnesium sulfate heptahydrate, 0.09 g/L zinc sulfate heptahydrate, and 15 g/L agar) at 30° C. for about a week.

(iv) Selection of Gene-Introduced Strain

Spores were collected from the fungal cells cultured and fungal cells were isolated by using an inorganic agar medium (20 g/L glucose, 1 g/L ammonium sulfate 0.6 g/L potassium dihydrogen phosphate, 0.25 g/L magnesium sulfate heptahydrate, 0.09 g/L zinc sulfate heptahydrate, and 15 g/L agar) adjusted to pH3. The mycelia of the strain grown was partly scraped off by a toothpick, suspended in 10 mM Tris-HCl (pH8.5) and incubated at 95° C. for 10 minutes. Thereafter, the suspension was appropriately diluted with 10 mM Tris-HCl (pH8.5) and used as a genomic template solution for colony PCR. The colony PCR was carried out by using the above genomic template solution, primers NK-069 (SEQ ID No: 50) and NK-118 (SEQ ID No: 51) and KOD FX Neo (TOYOBO). The colony PCR using the above primers amplifies a DNA fragments if the trpC gene fragment was knocked-in at the pdc1 genetic locus. The strain, in which a DNA fragment was amplified by colony PCR, was obtained as a pdc1 gene-deficient strain, A pdc strain. The strain into which the gene was introduced by using plasmid pUC18-Ppdc-trpC-Padh-RdME1-Tpdc was designated as Δpdc:ME1 strain, whereas the strain into which the gene was introduced by using plasmid ptrpC-knock-in was designated as Δpdc:trpC strain. The remaining strains were scraped off by using a platinum loop and vigorously mixed in a spore collection solution (8.5 g/L sodium chloride, and 0.5 g/L polyoxyethylene sorbitan monooleate). After mixing, the spore suspension obtained was filtered through a glass filter 3GP100 cylindrical funnel type (Shibata Scientific Technology Ltd.) to obtain a spore liquid. The number of spores in the spore liquid was counted by using TC20 Automated Cell Counter (Biorad).

### Example 2 Measurement of Malic Enzyme Activity of Mutant Strain

### (1) Culture of Strain

(i) Preparation of Mycelia

A 500 mL Erlenmeyer flask with a baffle (Asahi Glass Co., Ltd.) was charged with 200 mL of SD/−Trp medium (Clontech) containing sorbitan monolaurate (Reodol SP-L10 (Kao Corp.)) at a final concentration of 0.5% (v/v). Each of the spore solutions of Δpdc:ME1 strain and Δpdc:trpC strain prepared in Example 1 was inoculated at 1×10³ spores/mL (medium) and then cultured at 27° C. for 3 days while stirring at 170 rpm. The resultant cultured broth was filtered through a stainless sieve (mesh size: 250 μm (AS ONE Corporation)) previously sterilized to collect fungal cells on the filter.

(ii) Proliferation of Mycelia

To 100 mL of an inorganic culture solution (0.1 g/L (NH₄)₂SO₄, 0.6 g/L KH₂PO₄, 0.25 g/L MgSO₄.7H₂O, 0.09 g/L ZnSO₄.7H₂O, 50 g/L calcium carbonate, and 100 g/L glucose) charged in a 500 mL Erlenmeyer flask, 5.0 to 8.0 g of the wet fungal cells collected in (i) was inoculated and cultured at 27° C. for about 40 hours, while stirring at 220 rpm. The resultant cultured broth was filtered by using a stainless screen filter holder (MILLIPORE) previously sterilized to collect fungal cells on the filter. The fungal cells were further washed with 200 mL of physiological saline on the filter holder. The physiological saline used for washing was removed by suction filtration.

(2) Preparation of Homogenate of Fungal Cells

6.0 g of fungal cells obtained in (1) in the above was inoculated in 40 mL of an inorganic culture solution (0.0175 g/L (NH₄)₂SO₄, 0.06 g/L KH₂PO₄, 0.375 g/L MgSO₄.7H₂O, 0.135 g/L ZnSO₄.7H₂O, 50 g/L calcium carbonate, and 100 g/L glucose) charged in a 200 mL Erlenmeyer flask and cultured at 35° C. for 24 hours while stirring at 170 rpm. The obtained cultured broth was filtered by a stainless screen filter holder (MILLIPORE) previously sterilized and fungal cells were collected on the filter. The fungal cells were further washed with 200 mL of physiological saline on the filter holder and the physiological saline was removed by suction filtration.

The fungal cells were divided into 1.0 g aliquots and each were frozen at −80°. The frozen fungal cells were homogenized by using a multi bead shocker and metal cone (Yasui Kikai Corporation). To this, 1 mL of 50 mM Tris-HCl buffer (pH8.0) was added and the resultant was homogenized again and centrifuged at 15,000 rpm and 4° C. for 5 minutes. Thereafter, the supernatant was concentrated by using AmiconUltra-0.5 (3 kDa, MILLIPORE) and washed to obtain a homogenate of fungal cell.

(3) Measurement of Malic Enzyme Activity

To a 96-well assay plate (Iwaki) to which 5 μL of the homogenate of fungal cell obtained in the above (2) was added, 185 μL of a reaction solution (final concentration 50 mM Tris-HCl pH8.0, 2.5 mM MnCl₂, and 0.2 mM NAD⁺) was added. A reaction was initiated by adding 10 μL of 200 mM malic acid. Based on the slope of absorbance change (NADH extinction coefficient=6200 M⁻¹ cm⁻¹) at 340 nm and 30° C., an activity value (mU/cell wet weight g) was obtained by calculation. The activity unit (U) herein was defined as the amount of malic acid consumed per minute (μmol/min). The measurement results are shown in Table 2. The malic enzyme activity of Δpdc:ME1 strain was improved about three times as large as that of a reference strain, Δpdc:trpC strain.

TABLE 2

| | Malic enzyme activity | |
|---|---|---|
| Sample | (mU/cell wet-weight g) | Relative activity |
| Δpdc::trpC strain | 2.5 | 1 |
| Δpdc::ME1 strain | 7.1 | 2.8 |

### Example 3 C4 Dicarboxylic Acid Productivity of ΔPdc:ME1 Strain

### (1) Culture of Strain

The mycelia were prepared and allowed to proliferate in the same conditions as in Example 2 (1).

### (2) Evaluation of C4 Dicarboxylic Acid Productivity of Transformed Strain

6.0 g of wet fungal cells of Δpdc:ME1 strain and Δpdc:trpC strain obtained in (1) in the above were inoculated in 40 mL of an inorganic culture solution (0.0175 g/L (NH₄)₂SO₄, 0.06 g/L KH₂PO₄, 0.375 g/L MgSO₄.7H₂O, 0.135 g/L ZnSO₄.7H₂O, 50 g/L calcium carbonate, and 100 g/L glucose) charged in a 200 mL Erlenmeyer flask and cultured at 35° C. while stirring at 170 rpm. After culture for 8 hours, the culture supernatant containing no fungal cell was collected and subjected to quantification of C4 dicarboxylic acid (fumaric acid) in accordance with the procedure of Reference Example 1 described later. Based on the obtained amount of C4 dicarboxylic acid, the improvement rate of C4 dicarboxylic acid productivity in Δpdc:ME1 strain was calculated in accordance with the following expression.

Improvement rate (%)=(production speed in Δpdc:ME1 strain/production speed in Δpdc:trpC strain)×100−100

The results are shown in Table 3. It was observed that the fumaric acid productivity of Δpdc:ME1 strain was improved by 40%, compared to that of Δpdc:trpC strain in which RdME1 gene had not been introduced.

TABLE 3

| | Production | Improvement rate of |
|---|---|---|
| Name of strain | speed (g/L/h) | productivity (%) |
| Δpdc::trpC strain | 1.5 | — |
| Δpdc::ME1 strain | 2.1 | 40 |

### Reference Example 1 Quantification of C4 Dicarboxylic Acid

A C4 dicarboxylic acid in a culture supernatant was quantified by HPLC. The culture supernatant to be subjected to HPLC analysis was appropriately diluted in advance with 37 mM sulfuric acid. Thereafter, insoluble matter was removed by using DISMIC-13cp (0.20 μm cellulose acetate membrane, ADVANTEC) or AcroPrep 96 filter plate (0.2 μm GHP membrane, Pall Corporation).

As an HPLC apparatus, LaChrom Elite (Hitachi High-Technologies Corporation) was used. As an analysis column, a polymer column for organic acid analysis, ICSep ICE-ION-300 (7.8 mm I.D.×30 cm, TRANSGENOMC) to which ICSep ICE-ION-300 Guard Column Cartride (4.0 mm I.D.×2.0 cm, TRANSGENOMIC) was connected was used. As an eluent, 10 mM sulfuric acid was used. Elution was carried out at a flow rate of 0.5 mL/minute and at a column temperature of 50° C. The C4 dicarboxylic acid was detected using a UV detector (detection wavelength 210 nm). A concentration calibration curve was prepared by using a standard sample [fumaric acid (distribution source code 063-00655, Wako Pure Chemical Industries, Ltd.)]. Based on the concentration calibration curve, the C4 dicarboxylic acid in the culture supernatant was quantified.

A value obtained by subtracting the initial amount of the C4 dicarboxylic acid in the medium from the amount of C4 dicarboxylic acid quantified in the medium was regarded as the amount of C4 dicarboxylic acid produced. A value obtained by dividing the amount of C4 dicarboxylic acid per culture medium at 8 hours after the start of culture by culture time was regarded as the production speed of C4 dicarboxylic acid by the cell.

1. A mutant filamentous fungus having enhanced expression of at least one polypeptide selected from the group consisting of the following:
a polypeptide consisting of the amino acid sequence of SEQ ID No: 2,
a polypeptide consisting of an amino acid sequence having an identity of at least 90% with the amino acid sequence of SEQ ID No: 2 and having malic enzyme activity, and
a polypeptide consisting of an amino acid sequence having a deletion, substitution, addition or insertion of one or more amino acids with respect to the amino acid sequence of SEQ ID No: 2 and having malic enzyme activity.

2. The mutant filamentous fungus according to claim 1, comprising a DNA fragment or vector introduced therein, wherein the DNA fragment or vector comprises at least one polynucleotide selected from the group consisting of the following:
a polynucleotide consisting of the nucleotide sequence of SEQ ID No: 1,
a polynucleotide consisting of a nucleotide sequence having an identity of at least 90% with the nucleotide sequence of SEQ ID No: 1 and encoding a polypeptide having malic enzyme activity, and
a polynucleotide consisting of a nucleotide sequence having a deletion, substitution, addition or insertion of one or more nucleotides with respect to the nucleotide sequence of SEQ ID No: 1 and encoding a polypeptide having malic enzyme activity.

3. The mutant filamentous fungus according to claim 1, wherein the filamentous fungus is Rhizopus.

4. A production method for a C4 dicarboxylic acid, comprising culturing the mutant filamentous fungus according to claim 1.

5. The production method according to claim 4, further comprising collecting the C4 dicarboxylic acid from the cultured broth.

6. The production method according to claim 4, wherein the C4 dicarboxylic acid is fumaric acid, malic acid or succinic acid.

7. A method for producing a mutant filamentous fungus, comprising enhancing, in a host filamentous fungus, expression of at least one polypeptide selected from the group consisting of the following:
a polypeptide consisting of the amino acid sequence of SEQ ID No: 2,
a polypeptide consisting of an amino acid sequence having an identity of at least 90% with the amino acid sequence of SEQ ID No: 2 and having malic enzyme activity, and
a polypeptide consisting of an amino acid sequence having a deletion, substitution, addition or insertion of one or more amino acids with respect to the amino acid sequence of SEQ ID No: 2 and having malic enzyme activity.

8. The method according to claim 7, wherein the enhancing expression comprises introducing a DNA fragment or vector containing at least one polynucleotide selected from the group consisting of the following:
a polynucleotide consisting of the nucleotide sequence of SEQ ID No: 1,
a polynucleotide consisting of a nucleotide sequence having an identity of at least 90% with the nucleotide sequence of SEQ ID No: 1 and encoding a polypeptide having malic enzyme activity, and
a polynucleotide consisting of a nucleotide sequence having a deletion, substitution, addition or insertion of one or more nucleotides with respect to the nucleotide sequence of SEQ ID No: 1 and encoding a polypeptide having malic enzyme activity.

9. The method according to claim 7, wherein the filamentous fungus is Rhizopus.

10. The mutant filamentous fungus according to claim 2, wherein the filamentous fungus is Rhizopus.

11. A production method for a C4 dicarboxylic acid, comprising culturing the mutant filamentous fungus according to claim 2.

12. The production method according to claim 11, further comprising collecting the C4 dicarboxylic acid from the cultured broth.

13. The production method according to claim 11, wherein the C4 dicarboxylic acid is fumaric acid, malic acid or succinic acid.

14. The method according to claim 8, wherein the filamentous fungus is Rhizopus.

15. A production method for a C4 dicarboxylic acid, comprising culturing the mutant filamentous fungus according to claim 3.

16. The production method according to claim 15, further comprising collecting the C4 dicarboxylic acid from the cultured broth.

17. The production method according to claim 16, wherein the C4 dicarboxylic acid is fumaric acid, malic acid or succinic acid.
